(19) United States
(12) Patent Application Publication
(10) Pub. No.: US 2024/0053354 A1
(43) Pub. Date: Feb. 15, 2024

(54) FUNCTIONAL POROSOME MANIPULATION

(71) Applicant: Porosome Therapeutics, Inc., Boston, MA (US)

(72) Inventors: Won Jin Cho, Newton, MA (US); Bhanu P. Jena, Bloomfield Hills, MI (US)

(21) Appl. No.: 18/446,111

(22) Filed: Aug. 8, 2023

Related U.S. Application Data
(63) Continuation-in-part of application No. 18/222,784, filed on Jul. 17, 2023.
(60) Provisional application No. 63/396,040, filed on Aug. 8, 2022.
(60) Provisional application No. 63/431,946, filed on Dec. 12, 2022.
(60) Provisional application No. 63/444,451, filed on Feb. 9, 2023.
(60) Provisional application No. 63/447,371, filed on Feb. 22, 2023.
(60) Provisional application No. 63/458,471, filed on Apr. 11, 2023.
(60) Provisional application No. 63/459,762, filed on Apr. 17, 2023.
(60) Provisional application No. 63/472,674, filed on Jun. 13, 2023.
(60) Provisional application No. 63/523,970, filed on Jun. 29, 2023.
(60) Provisional application No. 63/526,786, filed on Jul. 14, 2023.

(51) Int. Cl. G01N 33/68

(52) U.S. Cl. CPC G01N 33/6845; G01N 33/6848

(57) ABSTRACT

The porosome is the main secretory structure of the eukaryotic cell. Presented herein are compositions and methods for the control and regulation of the porosome structure. Including a method of porosome-associated-protein and interacting small molecule identification; usage of small molecules targeted to one or more porosome proteins; compositions and usages of nanobodies coupled with small molecules; the reconstitution of porosomes; and the creation and usage of artificial porosome structures.

### CROSS REFERENCE TO RELATED APPLICATIONS

This application claims the benefit of U.S. Prov. Pat. App. No: 63/396,040 filed on 8 Aug. 2022; U.S. Prov. Pat. App. No: 63/431,946 filed on 12 Dec. 2022; U.S. Prov. Pat. App. No: 63/444,451 filed on 9 Feb. 2023; U.S. Prov. Pat. App. No: 63/447,371 filed on 22 Feb. 2023; U.S. Prov. Pat. App. No: 63/458,471 filed on 11 Apr. 2023; U.S. Prov. Pat. App. No: 63/459,762 filed on 17 Apr. 2023; U.S. Prov. Pat. App. No: 63/472,674 filed on 13 Jun. 2023; U.S. Prov. Pat. App. No: 63/523,970 filed on 29 Jun. 2023; and U.S. Prov. Pat. App. No: 63/526,786 filed on 14 Jul. 2023, the entireties of which are hereby incorporated by reference. The application is a further CIP of pending U.S. patent application Ser. No. 18/222,784 filed 17 Jul. 2023, the entirety of which is incorporated herein by reference.

### TECHNICAL FIELD

This disclosure provides methods and compositions for modulating, including regulating or altering, the structure and function for the porosome organelle and/or its component proteins. In certain embodiments, the porosome modulation methods can alter disease progression. In some embodiments, an identified small molecule modulator, or a pharmaceutically acceptable salt or solvate thereof can be used to modulate the activity of one or more porosome proteins, affecting the structure and function of the porosome organelle. Also disclosed are methods of using such targeted molecules for treating porosome related defects. Additional embodiments include functional reconstitution of porosome, or porosome-like, structures in target cells to overcome physiological defects. Additional embodiments further include the usage of humanized nanobodies alone, or in combination with small molecules to target one or more porosome proteins to effect a change in the structure and/or function of a porosome complex.

### DISCUSSION OF ART

Porosome organelles are cup-shaped supramolecular lipoprotein structures located at the cell plasma membrane. They are the sites at which secretory vesicles inside the cell transiently dock, fuse, and secret their contents outside the cell.

Typical porosome structures range in size from 15 nm in neurons to 100-180 nm in endocrine and exocrine cells. Porosomes are composed of about 30-40 proteins, with the porosome composition depending on cell type. Porosome-mediated secretion across the cell plasma membrane is a fundamental process through which cells communicate with their environment and exchange information. In a multicellular context, porosome secretion enables cell communities to communicate and maintain homeostasis and, thus, sustain life. Porosomes are present in all secretory cells, from the digestive enzyme-secreting pancreatic acinar cells to the hormone-releasing growth hormone and insulin-secreting cells, mast cells, chromaffin cells, hair cells of the inner ear, and in neurons secreting neurotransmitters. Porosomes have been immunoisolated from a number of cells including the insulin-secreting beta cells of the exocrine pancreas, cells of the human airways epithelia and neurons, biochemically characterized, and functionally reconstituted into artificial lipid membranes. A large body of evidence has accumulated on the role of porosome-associated proteins on cell secretion and secretory defects, including: neurotransmission and neurological disorders; respiratory disorders; and insulin secretion disorders. Thus, defects in cell secretion stemming from porosome, or porosome component, malfunction are implicated to underpin numerous disease mechanisms including those for cystic fibrosis, diabetes, Alzheimer's, Down Syndrome, schizophrenia, digestive, and immune disorders, among others.

Whereas the “parts list” of biology has been relatively well defined, with many proteins identified in the human cell, a systems level understanding of disease modalities has only begun to be appreciated fully. It is no longer correct to state that any given disease results from a bad copy of a single protein; or, in the face of epigenetics, that expression of a given protein structure results from a single gene alone. In addition, numerous proteins are found to collectively participate in multiple cellular processes (e.g., usually expressed as found in different metabolic or enzymatic pathways), making targeting of single proteins difficult to do without altering multiple cellular functions.

Therefore, targeting a single protein, either for binding or degradation, may treat a disease, but may also inhibit essential bodily functions and/or alternative biochemical pathways that require that same protein. This leads to adverse side effects. Furthermore, each protein requires a “groove” or “binding pocket” for a putative drug to grab onto to bind. Furthermore, this binding pocket on a target protein is also influenced by its neighboring proteins, hence the entire complex needs to be carefully understood for appropriate therapy. Additionally, for small molecules, it is estimated that only 20%-25% of proteins have the necessary groove to bind and be modulated, inhibited or activated.

Given the great demand imposed by the nature of the diseases caused by secretory function defects, there is a great need for methods that modulate porosome structure or function in order to correct or otherwise modify secretory defects; and, consequently, treat diseases resulting from porosome secretory defects. Further, in cases where porosome structures are defective or absent, there is a need to restore the porosome structure or provide a porosome or porosome-like structure to an affected cell.

### SUMMARY

This disclosure provides a method for identifying protein interactions that modulate porosome function or restore the function of specific porosome proteins. In some embodiments high-throughput chemical screening techniques are employed. In still other embodiments, in silico techniques are employed. Identified proteins are subsequently validated for activity and function in animal models and, eventually, in humans. In certain embodiments the method entails: Creating a first porosome sample mixture; said porosome sample mixture is then incubated with a labeling group generating a probe-protein complex. The probe-protein complex is then harvested and fragmented, resulting in protein fragments. The protein fragments are then analyzed via a proteomic method. Proteins in the porosome sample mixture may then be identified, creating a first identified protein set. A value is assigned to each protein in the first identified protein set. In some instances, the assigned value may be indicative of the quantity of the protein in the porosome sample mixture either in absolute (e. g., grams or moles) or relative (e.g., percent or ratio) terms. The above steps are repeated for a second sample porosome mixture, obtaining a second value for each protein in a second identified protein set. A ratio between the values of paired proteins in the first and second identified protein sets is then calculated. In some instance the first value is ratioed to the second; in others, the second is ratioed to the first. The obtained ratio is thus determinative of a protein-protein interaction either within or adjacent to a porosome structure.

In certain embodiments, the first sample porosome mixture is from a standard, control, or wildtype cell sample and the second sample porosome mixture is from a test cell sample. In addition, the test cell sample may be a knock-out cell line.

In still other embodiments, the first and second porosome cell sample mixtures may be at least one selected from the group of: a cell sample, a cell lysate sample, and isolated porosome proteins.

In still other embodiments of the above method, the probe-protein complex is conjugated to a chromophore during the incubation step. In still other embodiments the probe-protein complex, or a subsample thereof, is separated and visualized via electrophoresis after incubation and before harvest of the probe-protein complex.

In certain other embodiments of the above method, fragmentation is completed or accomplished via at least one selected from the group of mechanical stress, pressure, and a chemical fragmentation agent. In still other embodiments, the chemical fragmentation agent is a protease.

In still other embodiments of the above method, the proteomic method includes usage of mass spectrometry. Indeed, in still other embodiments the proteomic method is at least one selected from the group of: LC, LC-MS, MALDI-TOF, GC-MS, CE-MS, and NMR. Further, in additional embodiments, the value for each protein in the first and second identified protein sets is obtained from a mass-spectroscopy analysis. In additional embodiments, the value is the area-under-the-curve from a plot of signal intensity as a function of mass-to-charge ratio. In additional embodiments, the value for each protein in the first and second identified protein sets correlates with the reactivity of a Lys residue within a protein. In additional embodiments the identified protein-protein interactions may be confirmed using a small molecule. The confirmation may be performed via a chemical cross-linkage and subsequent confirmation of linkage via mass spectrometry.

In certain embodiments, the functioning of porosomes from the first and second samples may be examined in an artificial lipid bilayer membrane.

In still other embodiments, an artificial porosome may be created and constructed in an artificial lipid bilayer membrane.

For all of the above, a kit may be created to accomplish one or more steps of the method.

The disclosure includes a method of modulating prosome activity in a subject in need thereof, for example by administering an effective amount of an identified small molecule to the subject.

In some embodiments, an identified small molecule modulator or a pharmaceutically acceptable salt or solvate thereof is used to modulate the activity of one or more porosome proteins.

In some embodiments the porosome proteins can be, for example, neuronal porosome proteins; mucus secreting porosome proteins of airway epithelia; insulin secreting proteins; digestive enzyme secreting; or signal molecule secreting proteins.

In some embodiments, the disclosure provides small molecules, e.g. a molecule of less than 1000 MW, that can target and regulate the production of one or more porosome constituent proteins by regulating, modulating, controlling the expression of, the methylation state of, or interfering with (e.g., RNAi, siRNA) the genomic sequence (DNA, RNA) encoding the constituent proteins. In some embodiments host cells are harvested from a patient, treated with a small molecule to alter porosome structure or function, and then returned to a patient.

In some embodiments, exosome release is controlled via the altered structure and/or functioning of the porosome complex or porosome-associated proteins. In certain embodiments one or more cell types may have genes of putative porosome complex or porosome-associated proteins “knocked out” through the use of CRISPER, RNAi, or other methods such as are known in the art.

In certain embodiments, a proteome and lipidome database is cross-correlated to identify candidate interaction sites. Protein-protein or protein-lipid candidates are chemically cross-linked and evaluated via mass spectrometry to confirm the interaction. Upon confirmation of the interaction a multivalent small molecule is created. Such a multivalent molecule may, for example, contain one or more peptide target sequences attached to a cross-linking molecule. Such a cross-linking molecule is further configured to hold the peptide target sequences at the correct orientation and distance so as to allow the peptide target sequences to bind to the at least two porosome-associated targets resulting in a modulation of porosome function. Validation of porosome function modulation is then carried out via standard drug validation pathways from animal models through to human clinical trials. For example, validation may first be done by assessment in-silico of the binding affinities to the target porosome proteins and lipids, followed by cell culture, organoid and animal studies.

In certain embodiments, isolated porosomes from a certain tissue or cell type in an artificial lipid bilayer membrane can also be used to screen, optimize the dose, or determine the efficacy of candidate drugs. In certain embodiments, isolated porosomes in an artificial lipid bilayer membrane may be administered to a subject suffering from a porosome-mediated disease (e.g., a disease where secretory function alteration is known or implied, such as, cystic fibrosis, diabetes, Alzheimer's, and the like).

Some embodiments relate to a method of modulating prosome activity in a subject in need thereof. In such embodiments an effective amount of an identified small molecule is administered to a subject in need thereof.

In some embodiments, an identified small molecule modulator or a pharmaceutically acceptable salt or solvate thereof may be used to modulate the activity of one or more porosome proteins.

In some embodiments, the porosome proteins may be neuronal porosome proteins. In still other embodiments the porosome proteins may be from airway epithelial cells. In still other embodiments the porosome proteins may be insulin secreting proteins. In still other embodiments, the proteins may be digestive enzyme secreting. In still other embodiments, the porosome proteins may be signal molecule secreting proteins from signal molecule secreting cells.

In some embodiments, one or more small molecules may target and regulate the production of one or more porosome constituent proteins by regulating, modulating, controlling the expression of, the methylation state of, or interfering with (e.g., RNAi, siRNA), one or more amino-acid chains (DNA, RNA) encoding the constituent proteins. In some embodiments, host cells may be harvested from a patient, incubated, expanded, purified, treated with a small molecule to alter porosome structure or function, and then returned to a patient.

In still other embodiments the disclosure provides an engineered nanobody that is used alone or in conjunction with one or more small molecules to alter the function of a porosome. For example, a nanobody targeted to a first porosome protein binding site, can be used in combination with one or more small molecules that interact with one or more additional porosome proteins. In still other embodiments an artificial porosome is cross-linked to a nanobody humanized to bind to one or more domains of one or more porosome or porosome-associated proteins. The cross-linked artificial porosome nanobody may then be delivered to a subject.

In still other embodiments there is provided a composition of at least one cross-linking molecule and at least one small molecule modulator targeted to a porosome protein. In certain embodiments the cross-linking molecule is an ELP deblock. In still other embodiments the cross-linking molecule is p-acetyl phenylalanine. In still other embodiments, the cross-linking molecule is maleimide. In still other embodiments the small molecule modulator is CDN1163. In still other embodiments there is at least a second small molecule modulator. In still other embodiments the second small molecule modulator is targeted to a porosome lipid. In certain embodiments the targeted porosome protein is at least one selected from the group of: syntaxin-1A, SNAP-25, SNAP-23, and actin. In still other embodiments the small molecule modulator is one targeted to a protein identified using the above-described method. In still other embodiments there is provided a nanobody humanized to target and bind to one or more domains of one or more porosome proteins. In still other embodiments, the humanized nanobody contains an artificial cysteine configured to enable attachment of an ELP deblock to the nanobody. In still other embodiments, the nanobody is further linked to one or more small molecules, forming a multivalent structure.

In still other embodiments, this disclosure provides for a humanized nanobody with one or more small molecules targeting the domains of one or more porosome proteins and an artificial cysteine. An ELP diblock is bound to the cysteine and to pAcF. In certain embodiments a drug is attached to the pAcF. In still other embodiments the drug attached to the pAcF is doxorubicin. In still other embodiments, the one or more domains of the one or more porosome proteins are identified to interact with one or more porosome proteins found to interact with each other. In yet other embodiments, the one or more domains additionally comprise at least one selected from the group of: K+ channels, aquaporin water channels, anion exchangers, membrane fusion proteins, sodium bicarbonate transporters, Gαi3, syntaxin-1A, SNAP-25, SNAP-23, and actin.

This disclosure further provides a method comprising the extraction of porosomes from a non-human source. The porosomes are then reconstituted into a human cell. In some embodiments, the porosomes are extracted from human epithelial cells and/or stem cells. In still other embodiments of the method, the extracted porosomes are reconstituted into organoids or an artificial lipid bilayer. In still other embodiments the non-human source is a pig or other mammal.

This disclosure also provides a method of identifying small molecules that target specific porosome proteins to modulate or restore porosome function. In some embodiments the single CRISPR knockout of selected porosome proteins, is used to determine what additional proteins within the porosome complex are lost in addition to the knockout protein. Those additional proteins that are lost from the porosome complex are believed ones associated with each other and the knockout protein within the complex.

Some embodiments include a method of modulating porosome-mediated insulin secretion in a subject in need thereof. In such embodiments, the over expression of a porosome protein(s) effective to synthesize and secrete insulin on glucose challenge is induced in the subject and/or an effective amount of an identified small molecule to synthesize and secrete insulin on glucose challenge is administered to the subject. The porosome protein of this embodiment can be a porosome protein with the pancreatic β cells of the subject.

In some embodiments, overexpression of either insulin secreting porosome protein ATP2C1 (ATPase secretory pathway Ca2+ transporting) or APOa1 alone or together enables both the increased expression and secretion of insulin in β-cells of the endocrine pancreas.

In some embodiments, the Ca2+-ATPase activator CDN1163, is used to increase both the expression of insulin and its secretion in β-cells of the endocrine pancreas.

In certain embodiments the disclosure provides a method of reconstituting functional porosome complexes into live cells to ameliorate or correct secretory defects resulting in the malfunction of one or more porosome proteins. In certain embodiments the reconstitution is performed on neuronal cells or cells from the exocrine and endocrine pancreas. In still other embodiments, other cell types, include the mucin-secreting porosome complex in the lung epithelia in cystic fibrosis (CF) patients.

In another embodiment the disclosure provides a method for scaled-up isolation of the mucin-secreting porosome complex from human lung epithelia cells. In particular embodiments the method enables isolation of mucin-secreting porosome complexes from Calu3 and other epithelial cells for reconstitution therapy in CF patients. This prevents immune rejection and enables amelioration of secretory defects as a consequence of the malfunction of porosome-associated cystic fibrosis transmembrane conductance regulator (“CFTR”) protein.

In an additional embodiment this disclosure provides a method for identifying protein-protein interactions within functional porosome complexes in cells. In particular, there is disclosed identification of protein-protein interactions with the CFTR protein within the mucin-secreting porosome complex in the epithelia of airways. Thus, additional embodiments enable the ability to fine tune regulation of the mucin-secreting porosome secretory machinery in cells of the lung epithelia and its precision targeting using small molecule drug-nanobody complexes.

In an additional embodiment there is a method for identifying one or more modulator(s) of the mucin-secreting porosome protein(s), to optimize mucin production and secretion from cells of airway epithelia. These mucin-secreting porosome protein modulators are used alone or in combination with reconstituted porosome complexes in CF therapy.

This disclosure further provides an approach to appropriately identify and match small molecules to target specific porosome proteins to modulate or restore mucin-secreting porosome function. In some embodiments the CRISPR knockout of porosome proteins one at a time, is used to determine what other proteins within the complex are lost at the porosome complex in addition to the knockout protein, especially the CFTR protein. In certain embodiments, those proteins that are lost from the porosome complex when CFTR is knocked out are classified as ones associated with CFTR within the complex. In certain embodiments the identified associated proteins are targeted to modulate and ameliorate secretory function and correct CFTR-mediated secretory defects.

Some embodiments relate to a method of modulating prosome-mediated mucin secretion activity in a subject in need thereof. In such embodiments the effective reconstitution of the functional mucin-secreting and or an effective amount of an identified small molecule is administered to the airways epithelia cells of a subject in need thereof to assist in the appropriate secretion of mucin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Features and advantages of embodiments of the present invention will become apparent on reading the detailed description below with reference to the drawings, which are illustrative but non-limiting, wherein:

FIG. 1 (A-F) illustrates examples of porosome structure in exocrine pancreas, neurons, and growth hormone secreting cells of the pituitary gland under different imaging modalities.

FIG. 2 illustrates a schematic drawing of porosome-mediated secretion.

FIG. 3 illustrates a schematic representation of an embodiment of the present invention; the porosome complex and its precise targeting for therapy.

FIGS. 4A and B is a schematic drawing depicting the predicted interactions between identified proteins within a neuronal porosome proteome and other regulatory proteins.

FIG. 5 is an example of drug targeting specificity using a combinatorial small molecule drug design with tissue-specific and porosome-specific multivalent nanobodies.

FIG. 6 is depiction of porosome-associated proteins involved in different classes of diseases.

FIG. 7 A-C is an illustration of the knockout (KO) and overexpression (OE) of porosome proteins ATP2C1 and APOa1 in insulin-secreting Min6 cells.

FIG. 8 is an illustration of glucose-stimulated insulin secretion in knockout (KO) and over expression (OE) of three porosome proteins: ATP2C1, APOa1 and TREK1 in Min6 cells. APOa1 overexpression results in both increase in insulin synthesis and secretion.

FIG. 9 is an illustration of glucose-stimulated insulin secretion in in Min6 cells following a 2 h exposure to increasing concentrations of the ATP2C1 activator CDN1163. Ten micromolar CDN1163 is found to be the optimal dose.

FIG. 10 A-E presents representative electron micrographs of Calu-3 cells in culture demonstrating the presence of microvilli (MV) and 100 nm cup-shaped porosomes (P) at the cell plasma membrane.

FIG. 11 illustrates the use of Ussing chamber experiments showing forskolin-stimulated chloride release from Calu-3 cells which is inhibited in the presence of the CFTR inhibitor GlyH-101.

FIG. 12 A-F demonstrates CFTR inhibitors 172 and GlyH-101 inhibit forskolin-stimulated secretion of intra-vesicular mucin from Calu-3 cells.

FIG. 13 A-D presents transmission electron micrographs of porosome complexes associated with a docked secretory vesicle at the apical end of an exocrine pancreatic acinar cell.

FIG. 14 A-D presents transmission electron micrographs of insulin-secreting Min6 cells.

FIG. 15 A-D presents electron micrographs of reconstituted exocrine pancreatic porosome complexes in liposomes. Note the establishment of the cup-shaped porosomes once reconstituted.

FIG. 16 A-C illustrates that a lipid bilayer-reconstituted porosome complex from the exocrine pancreas is functional.

FIG. 17 A-C presents the enriched presence of TREK-1, Gi3, and Syntaxin-1A immunoreactivity in porosome reconstituted insulin-secreting Min6 cells.

FIG. 18 A-C illustrates insulin-secreting porosomes reconstituted in live Min6 cells, demonstrating elevated glucose-stimulated insulin secretion.

FIG. 19 A-B illustrates the enriched presence of G″i3, and Syntaxin-1A immunoreactivity and increased glucose-stimulated insulin secretion in porosome-reconstituted Min6 cells even after 48 h.

FIG. 20 is a schematic drawing of the delivery of a functional mucin-secreting porosome complex into the airway epithelia cells in CF patients and amelioration of CF disease symptoms.

FIG. 21 illustrates an immunoblot analysis of total Calu-3 cell homogenate (CH) and isolated porosome complex (P), demonstrates the presence of porosome proteins actin, Gαi3, and vimentin. Note the enriched presence of these proteins in the porosome complex.

FIG. 22 illustrates an immunoisolated CFTR-associated porosome complex using the CFTR-specific antibody. Note the pull-down of the porosome associated proteins such as syntaxin-1A (present as 70 kDa t-/v-SNARE complex), SNAP-25 (present as 70 kDa t-/v-SNARE complex), SNAP-23 (present as 68 kDa t-/v-SNARE complex), and actin.

FIG. 23 illustrates the restoration of mucus secretion by CDN1163 in ΔF508-CFTR Human CF Bronchial Epithelial Cells.

FIG. 24 illustrates increased mucus secretion by α-CPA in ΔF508-CFTR Human CF Bronchial Epithelial Cells.

FIG. 25 illustrates a schematic drawing demonstrating some interactions between CFTR and associated proteins.

### DETAILED DESCRIPTION OF THE INVENTION

### Identification of Porosome Protein Interaction Targets

Drug discovery in the modern era is the result of an evolution from past techniques; from screening for active substances from biologic extracts sourced directly from nature; to the advent of molecular biology techniques used to generate drugs directed against specific molecular targets; to the creation in the 1970s and 1980s of the modern drug development paradigm of “rational drug design” with a focus on small molecules and antibodies (used to block specific functions of a single protein target) and recombinant protein-based therapeutic agents. Subsequent breakthroughs in genomics and gene-editing technologies, stem cell biology, patient-derived organoids, cryo-electron microscopy, synthetic biology, and small molecule screening and image analysis using AI machine learning and high-throughput screening further transformed the structure of drug discovery. Indeed, it is through the assistance of AI models, such as AlphaFold2 and its kind, that the final folded structure of a given protein is predictable and made readily verifiable from a given genetic sequence. Further, such models are allowing for the development of so-called designer proteins not found in a natural setting.

Despite these breakthroughs, as pointed out by the National Cancer Institute, it remains a stunning fact that 85% of human proteins are not druggable (i.e., these proteins, which include disease-causing proteins, are not pharmacologically capable of being targeted). Indeed, it was pointed out that “the number of new drugs has been declining because it's becoming harder and harder to create new medicines. In other words, we've run out of proteins that can be targeted with drugs. The targets that are left are ‘undruggable.’ Most proteins that drive disease processes are undruggable.” (Brent Stockwell, Columbia University). The American Society of Clinical Oncology further notes that “pharmacologic targeting of intractable proteins is now a key challenge of modern drug development, requiring innovation and the development of new technologies.”

Attempts to address the formidable barrier of “undruggable” proteins to drug development include a select few innovative approaches. One approach is targeted protein degradation where a disease-causing protein is tagged for destruction, thus blocking its function. A second approach is the creation of a chemoproteomics platform used to identify previously unknown binding pockets in undruggable targets in an attempt to produce a variety of small molecule therapies across indications.

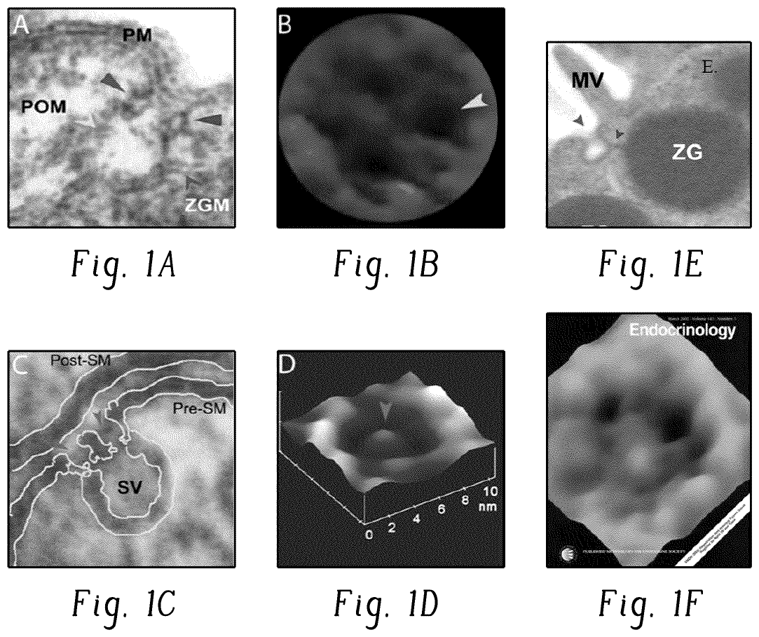

In stark contrast to the two above-outlined approaches, embodiments of the present invention encompass a wholly different third approach; one which takes a systems-level approach of targeting multiple proteins of the communication machinery of the cell as embodied by the porosome and its ability to alter cell secretory activity. This approach addresses specificity and precision targeting, while overcoming the “undruggable” problem and provides a pathway to uniquely address previously intractable conditions ranging from cystic fibrosis, diabetes to Alzheimer's, and to cancer caused by malfunctioning proteins and lipids involved in cell secretion. The present invention is unique in its pioneering focus on targeting both druggable and undruggable proteins and lipids that cause secretory defects and that, in turn, result in cystic fibrosis, cancers, neurological, endocrine and immunological disorders, among others. Thus, embodiments of the present invention control or alter the course of diseases by targeting multiple proteins within a target functional cellular complex (e.g., the porosome structure) that has malfunctioned. In certain embodiments, the target functional cellular complex is the porosome. In still other embodiments, multiple proteins within the porosome are targeted. In still other embodiments, one or more additionally identified proteins not found in the porosome are also identified and targeted. The virtue of such precise targeting results in the creation of a precision therapy with an expected overall reduction in treatment side-effects and an increase in therapeutic efficacy. FIG. 1 illustrates 1 illustrates examples of porosome structure in exocrine pancreas, neurons and neuroendocrine cells under different imaging modalities.

As shown in FIG. 1 (explained in further detail below), porosomes are cup-shaped supramolecular lipo-protein structures in the cell plasma membranes of eukaryotic cells. Secretory vesicles transiently dock and fuse with the porosome complex in the process of vesicle fusion and content release during secretion. Porosome structures are composed of many proteins which, themselves, often participate in other cellular complexes. Examples of purified neurosomal porosome proteins are presented in Table 1. Further examples of compositional porosomal proteins are available in the literature to those of ordinary skill in the art. Since the porosome is embedded in the lipid bilayer, porosome proteins may be wholly on one side or the other of the membrane or may be trans-membrane.

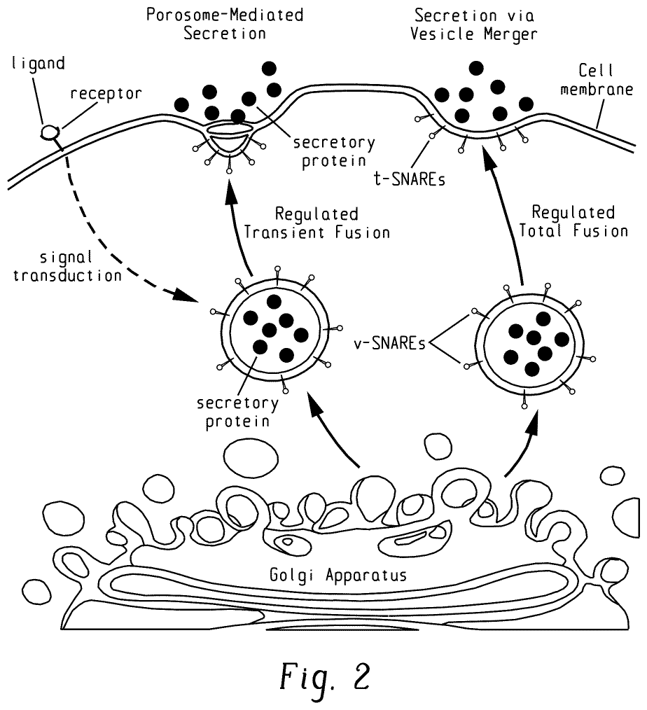

As illustrated in FIG. 2, the transient fusion of a secretory vesicle membrane at the porosome base via SNARE proteins, results in the formation of a fusion pore or continuity for the release of intra-vesicular contents from the cell. After secretion is complete, the fusion pore temporarily formed at the base of the porosome is resealed. The porosomes are few nanometers in size and contain many different types of protein, especially chloride and calcium channels, actin, and SNARE proteins that mediate the docking and fusion of the vesicles with the cell membrane. Once the secretory vesicles have docked with the SNARE proteins, they swell, which increases their internal pressure. They then transiently fuse at the base of the porosome, and these pressurized contents within secretory vesicles are ejected from the cell. Examination of cells following secretion using electron microscopy, demonstrate increased presence of partially empty vesicles following secretion. This suggests that during the secretory process, only a portion of the vesicular contents are able to exit the cell. This could only be possible if the vesicle were to temporarily establish continuity with the cell plasma membrane, expel a portion of its contents, then detach, reseal, and withdraw into the cytosol (endocytose). In this way, the secretory vesicle could be reused for subsequent rounds of exo-endocytosis, until completely empty of its contents. In a fast secretory cell like the neuron, neurotransmitter transporters present at the secretory vesicle membrane are able to refill the vesicles following their release during neurotransmission.

Porosomes vary in size depending on the cell type. Porosomes in the exocrine pancreas and in endocrine and neuroendocrine cells, for example, range from 100 nm to 180 nm in diameter while in neurons the porosomes range from 10 nm to 15 nm (about 1/10 the size of pancreatic porosomes). When a secretory vesicle containing v-SNARE docks at a porosome base containing t-SNARE, membrane continuity is established via the establishment of a t-/v-SNARE ring complex formed between the two opposing membranes. The size of the t/v-SNARE complex is directly proportional to the size of the vesicle. Secretory vesicles usually contain dehydrated proteins (non-active) which are activated once they are hydrated. GTP is required for the transport of water through the water channels or Aquaporins, and ions through ion channels to hydrate the vesicle prior to secretion. Once the vesicle fuses at the porosome base, the contents of the vesicle at high pressure are ejected from the cell.

Generally, the dynamics of opening of porosomes to the outside of the cell is regulated by actin and unconventional myosin; however, neurons requiring a rapid response have a central plug that moves vertically, enabling the opening and resealing the t-/v-SNARE induced continuity between the synaptic vesicle and the porosome base, to release neurotransmitters. Porosomes have been demonstrated to be the universal secretory machinery in cells. The neuronal porosome proteome and its detailed structure using solution x-ray has been solved, providing the composition and possible molecular architecture of the machinery. (Table 1)

TABLE 1
COMPOSITION AND POSSIBLE MOLECULAR
ARCHITECTURE OF THE MACHINERY

| GENE SYMBOL | MW | | PROTEIN NAME |
|---|---|---|---|
| ACTB | 42 | kDa | Actin, cytoplasmic 1 |
| AT1A3 | 112 | kDa | Sodium/potassium-transporting ATPase subunit alpha-3 |
| AT2B1 | 139 | kDa | Plasma membrane calcium-transporting ATPase 1 |
| AT2B2 | 137 | kDa | Plasma membrane calcium-transporting ATPase 2 |
| BASP1 | 22 | kDa | Brain acid soluble protein 1 |
| CAP1 | 52 | kDa | Adenylyl cyclase-associated protein 1 |
| CN37 | 47 | kDa | 2′,3′-cyclic-nucleotide 3′-phosphodiesterase |
| DPYL2 | 62 | kDa | Dihydropyrimidinase-related protein 2 |
| DPYL3 | 62 | kDa | Dihydropyrimidinase-related protein 3 |
| DPYL5 | 62 | kDa | Dihydropyrimidinase-related protein 5 |
| GLNA | 42 | kDa | Glutamine synthetase |
| GNAO | 40 | kDa | Guanine nucleotide-binding protein G(o) subunit alpha |
| NCAM1 | 95 | kDa | Neural cell adhesion molecule 1 |
| NSF | 83 | kDa | Vesicle-fusing ATPase |
| RAB3A | 25 | kDa | Ras-related protein Rab-3A |
| RTN3 | 102 | kDa | Reticulon-3 |
| RTN4 | 126 | kDa | Reticulon-4 |
| SNP25 | 25 | kDa | Synaptosomal-associated protein 25 |
| STX1A | 33 | kDa | Syntaxin-1A |
| STX1B | 33 | kDa | Syntaxin-1B |
| STXB1 | 68 | kDa | Syntaxin-binding protein 1 |
| SYN2 | 63 | kDa | Synapsin-2 |
| SYPH | 33 | kDa | Synaptophysin |
| SYT1 | 47 | kDa | Synaptotagmin-1 |
| TBA1A | 50 | kDa | Tubulin alpha-1A chain |
| VAMP1 | 13 | kDa | Vesicle-associated membrane protein 1 |
| VAMP2 | 13 | kDa | Vesicle-associated membrane protein 2 |
| VATB2 | 57 | kDa | V-type proton ATPase subunit B, brain isoform |

Examples of porosome structure are illustrated in FIG. 1, which depicts example porosomes in exocrine pancreas, neurons, and in growth hormone secreting cells of the pituitary gland. FIG. 1A is an electron micrograph of a single porosome at the apical plasma membrane (PM) of a pancreatic acinar cell showing the porosome membrane (POM, yellow arrowhead) associated with the membrane of a secretory vesicle called zymogen granule (ZGM). A circular ring structure (blue arrowhead), likely actin-myosin, forms the neck of the porosome complex. FIG. 1B is an atomic force microscope (AFM) micrograph of the apical surface topology of a live pancreatic acinar cell, demonstrating the presence of four openings or porosomes (one indicated by the yellow arrowhead). Porosomes in the exocrine pancreas range in size from 100-180 nm in diameter. FIG. 1C is an electron micrograph of a neuronal porosome (red arrowheads) with a docked synaptic vesicle (SV) at its base, in the presynaptic membrane (Pre-SM) of a nerve terminal. Note the central plug in the porosome complex. FIG. 1D is an AFM micrograph of a neuronal porosome at a presynaptic membrane in an isolated synaptosome. Note the central plug (red arrowhead). The neuronal porosome is an order of magnitude smaller (10-17 nm in diameter) in comparison to the porosome in the exocrine pancreas. FIG. 1E is an electron micrograph of porosome next to a microvilli (MV) at the apical plasma membrane (PM) of a pancreatic acinar cell with docked secretory vesicle or ZG. FIG. 1F is an AFM micrograph of the apical surface topology of a live GH cell from pig pituitary demonstrating the presence of 100-180 nm in diameter porosomes (black circular openings). Images are obtained from: Proc Natl Acad Sci 94:316-321 (1997); Biophys J 85:2035-2043 (2003); Cell Biol Int 28:699-708 (2004); J Microscopy 232:106-111 (2008); Endocrinology 143:1144-1148 (2003), the entireties of which are incorporated by reference for the teachings they contain.

As above mentioned, the “parts list” of biology is relatively well defined, with many proteins identified in the human cell. However, a systems level understanding of disease modalities has only begun to be appreciated fully. Numerous individual proteins participate in multiple cellular processes (e.g., are found in multiple enzyme activity pathways), making the targeting of a single protein type for the treatment of disease difficult to do without damaging multiple cellular functions. However, combinations of proteins (with their associated binding partners, coenzymes, etc.) offer a higher level of specificity for any particularly chosen biological processes. Embodiments of the invention provide the ability to selectively modulate a specific biological process by targeting a combination of proteins within a molecular complex that serves a specific biological function. This allows for the precise targeting of drugs at the “systems-level” physiology, inhibiting or stimulating cellular processes rather than individual protein or lipid molecules.

One such example of a systems-level cellular process is secretion through the porosome complex, the universal secretory machinery of the cell. Porosomes enable communication (language) between cells in the body by secreting chemical messages such as neurotransmitters from nerve cells or hormones from endocrine cells such as insulin from β-cells of the endocrine pancreas. These chemical messages are stored in secretory vesicles within the cell. The porosome secretory machinery, composed of 30+ proteins, provide instructions to secretory vesicles to appropriately dock at the porosome base, fuse, swell and release measured amounts of intra-vesicular contents to the outside. None of the individual components of the porosome are unique to it. Rather, the combination of 30 or more proteins together in the proper conformation within the complex, provides function to the structure. Thus, embodiments of the invention entail correcting cellular function by targeting a specific multimer, such as the porosome complex, without altering the activity of other cellular complexes, hence other cellular processes that possess one or more of the individual components of the targeted structure.

To be able to precisely target the proteins within the porosome complex, it is critical to obtain knowledge of protein-protein interactions within porosomes of a certain tissue or cell type. To accomplish this, embodiments of the present invention use an approach termed “interactomics.” Users of embodiments of the interactomics processes described herein can determine the protein-protein interactions within the porosome complex. Those of ordinary skill in the art may then be able to validate such interactions via CRISPR knockout of porosome proteins one at a time. Those using the skills taught herein along with standard techniques of the art may then determine what other proteins within a porosome complex are lost besides the knockout protein. We teach that those proteins that are additionally lost from the porosome complex are the ones associated with each other within the complex. Such systematic studies allow one to decipher the distribution of proteins within the porosome complex and help in targeting specific proteins within the complex to modulate and ameliorate secretory defects and the resulting disease.

In certain embodiments of the invention, a non-volatile computer-readable memory device executes a machine learning algorithm such as are known and trained on a database of small molecules cross-referenced to target multiple proteins within the porosome complex. Further embodiments make use of existing small molecules in a “bivalent” or “trivalent” form, presented using appropriate nanobody for targeted delivery. Such a drug delivery systems will help precision targeting to modulate protein function, only when bound to two or more protein targets of interest. Said nanobodies may further serve as the basis of cross-linking molecules.

By way of non-limiting example, supra paramagnetic iron oxide (SPIO) nanoparticles possess the ability for rapid entry and release of the cancer drug doxorubicin in cancer cells. Dextran-coated SPIO nanoparticle ferrofluid, functionalized with the red-auto-fluorescing doxorubicin and the green-fluorescent dye fluorescein isothiocyanate as a reporter, enables tracking the intracellular nanoparticle transport and drug release. This engineered nanoparticle enables a >20-fold rapid entry and release of the drug in human pancreatic cancer cells, holding therapeutic potential as an advanced drug delivery and imaging platform.

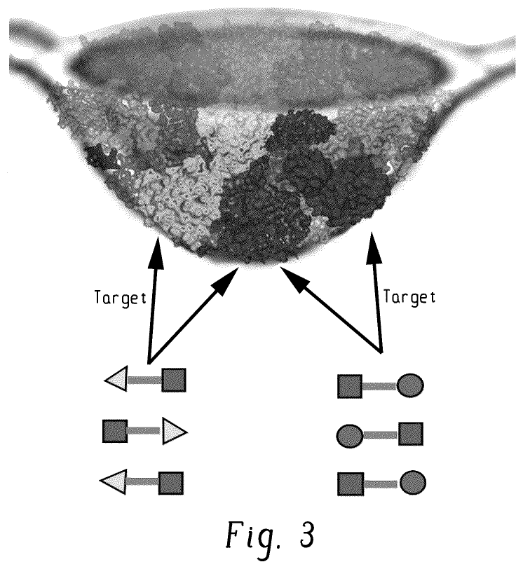

Like the letters of the alphabet that form meaningful words in the right combination, searching for a word using just one of its composing letters, would be nearly impossible. However, if two or more letters that compose the word are used in the search, particularly if the letters are in the sequence order used by the word, the likelihood of identifying the word is greatly enhanced. Further extending the metaphor in a general sense, and without being wholly bound to any single theory of operation; similarly, as schematically shown in FIG. 3 for an example porosome, when a drug is created to target and bind to multiple protein targets within a protein complex, the probability of the drug finding its specific cellular target complex, is greatly increased. In FIG. 3, for example, a hypothetical drug targeting multiple porosome proteins is created by the linkage of compounds (yellow triangles, red squares, blue circles) that interact with two or more protein targets adjacent to each other. Thus, a yellow triangle-red square combination my bind to the two adjacent yellow-red porosome proteins without affecting the blue porosome protein. Likewise, for the red square-blue circle combination not affecting the yellow protein. Thus, for the design and creation of such “targeted” type molecules it is important to elucidate and understand whether targeted proteins are adjacent to, or otherwise interact with, each other.

Each protein in the porosome complex (i.e., the “word”) is seen as a different letter in the alphabet. The correct association of each protein letter with the others, constitute the porosome word. Incorrect pairing or alterations in any of the protein letters, result in misspelling of the word porosome, leading to disease.

In an embodiment of the invention, candidate protein-protein interactions within a cellular functional complex, such as the porosome complex, are identified using the constantly updating STRING (Search Tool for the Retrieval of Interacting Genes/Proteins) database. STRING is a database of known and predicted protein-protein interactions. The interactions include direct (physical) and indirect (functional) associations; they stem from computational prediction, from knowledge transfer between organisms, and from interactions aggregated from other (primary) databases. At present the STRING database has at least 20 billion known protein-protein interactions between nearly 68 million proteins.

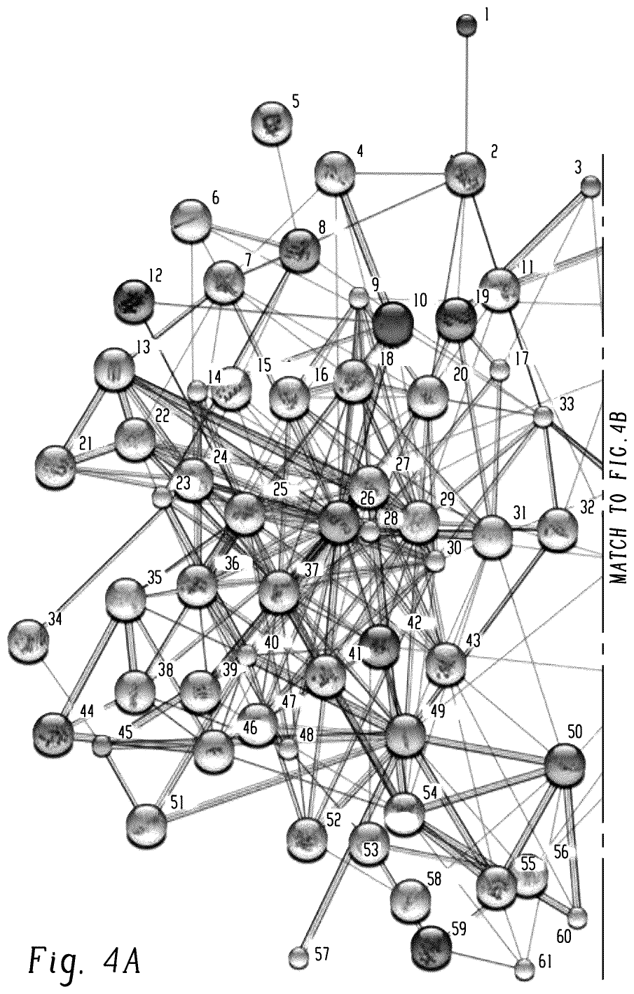

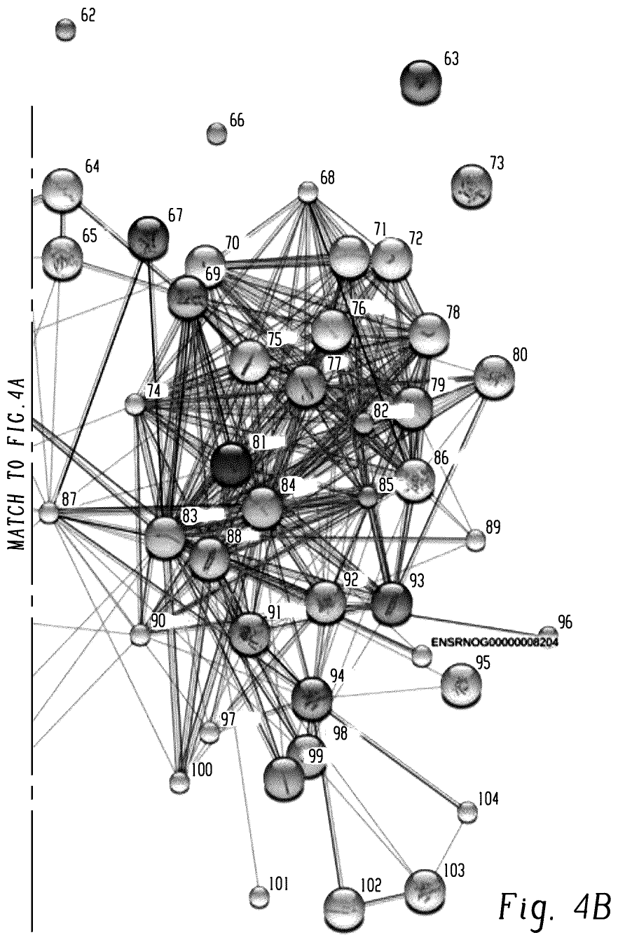

FIG. 4, for example, is a schematic drawing depicting the predicted interactions between identified proteins within a neuronal porosome proteome and other regulatory proteins. Due to space constraints, Table 2, below, lists the proteins within FIG. 4 as identified by numerical character. These interactions are generated from inputs of the identified proteins in the neuronal porosome, using STRING 9.0. STRING 9.0 is a database of known and predicted protein interactions. The interactions include direct (physical) and indirect (functional) associations derived from genomic, high-throughput, conserved co-expression, and earlier knowledge.

Illustrated are two clusters of protein-protein interactions identified in the porosome complex. The one cluster to the left, and most likely present at the apical end of the porosome cup are cytoskeletal structure and signaling proteins. The cluster to the right represents proteins that are primarily involved in membrane fusion including SNARE proteins and calcium channels, and therefore their location would be at the basal part of the porosome cup facing the cytosol. Interestingly, heterotrimeric GTP-binding protein and the GTP-binding membrane fission protein dynamin (Dnm2), are present in the left cluster. The presence of dynamin in the left cluster is of little surprise since they are microtubule-associated proteins, and intersectinl is also known to interact with dynamin. However, their involvement in fission of the neck of fused vesicles at the porosome base would require their presence at the base of porosomes. The confidence of the predicted functional interactions shown are >99%.

Identified protein-protein interactions of interest are then followed by chemical cross-linking with mass spectrometry used to: experimentally confirm the in silico interactions; further determine the interaction domains in 3D; and identify possible druggable sites. Thus, a practitioner of embodiments of the invention can design and present small molecules, or small molecules attached to carrier molecules, designed to bind simultaneously to multiple protein targets within a protein complex and functionally modulate the physiological function of the protein complex.

TABLE 2
FIG. 4 PROTEINS BY NUMBERS

| Number | Protein |
|---|---|
| 1 | Myh7b |
| 2 | Actb |
| 3 | Rtn3 |
| 4 | Tjp1 |
| 5 | Aqp4 |
| 6 | Des |
| 7 | Vcl |
| 8 | Dmd |
| 9 | Ntn1 |
| 10 | Gja1 |
| 11 | Grin2a |
| 12 | Tubb3 |
| 13 | Bcar1 |
| 14 | Mapk7 |
| 15 | Khdrbs1 |
| 16 | Rac1 |
| 17 | Omg |
| 18 | Cdc42 |
| 19 | Rtn4 |
| 20 | Wasl |
| 21 | Pipn1 |
| 22 | Crk |
| 23 | Ankz |
| 24 | Fn1 |
| 25 | Mapk2 |
| 26 | Src |
| 27 | Pipn11 |
| 28 | Mag |
| 29 | Fyn |
| 30 | Egf |
| 31 | Ngfr |
| 32 | Prng |
| 33 | Cav1 |
| 34 | Den |
| 35 | Citc |
| 36 | Mapk1 |
| 37 | Sos1 |
| 38 | Arrb1 |
| 39 | Sirpa |
| 40 | Licam |
| 41 | Hras |
| 42 | Sptbn2 |
| 43 | Syk |
| 44 | Dnm2 |
| 45 | Ncan |
| 46 | Sh3gt2 |
| 47 | Ptpra |
| 48 | F2r |
| 49 | Ncam1 |
| 50 | Gdnf |
| 51 | Cntn2 |
| 52 | Fgfr1 |
| 53 | Gnb1 |
| 54 | Ref |
| 55 | Gfra1 |
| 56 | Pich2 |
| 57 | StBsia2 |
| 58 | Bmp2 |
| 59 | Gnao1 |
| 60 | Gfra2 |
| 61 | Ednrb |
| 62 | Basp1 |
| 63 | Cap1 |
| 64 | Gria2 |
| 65 | Pick1 |
| 66 | Cd207 |
| 67 | Syn2 |
| 68 | Bet1 |
| 69 | Nsf |
| 70 | Stx7 |
| 71 | Vb1a |
| 72 | 5tx8 |
| 73 | Ckmt1 |
| 74 | Stx2 |
| 75 | Cptx2 |
| 76 | Vamp3 |
| 77 | Stx3 |
| 78 | Vamp8 |
| 79 | Stx4 |
| 80 | Stxbp3 |
| 81 | Vamp1 |
| 82 | Snap23 |
| 83 | Snap25 |
| 84 | Vamp2 |
| 85 | Napa |
| 86 | Vamp7 |
| 87 | Syp |
| 88 | Stx1a |
| 89 | Snapap |
| 90 | Snap29 |
| 91 | Stxbp1 |
| 92 | Syt1 |
| 93 | Stx1B |
| 94 | Rab3a |
| 95 | Syt4 |
| 96 | Cacna1b |
| 97 | Slc17a7 |
| 98 | Rpf3a |
| 99 | Stxbp5 |
| 100 | Cptx1 |
| 101 | Sic6a12 |
| 102 | Rph3al |
| 103 | Rab3c |
| 104 | Rab3ip |

Identified small molecules as candidate drugs passing the above-described process can then be further validated following the traditional pathways of studies on cell cultures, organoids and animals to determine drug suitability (safety, efficacy and stability); following which the candidate drugs would undergo clinical trials.

In an example of the above-described method, the mass spectrometry was performed on immune-isolated porosome complexes from human bronchial epithelial cell lines, namely control WT-CFTR Human Bronchial Epithelial Cell Line (CFBE41o-6.2), serving as a control or wild-type cell sample, and experimental ΔF508-CFTR Human CF Bronchial Epithelial Cell Line (CFBE41o) a ΔF508del (−/−) homozygous deletion serving as a test cell sample. The proteins that were identified and were associated with the porosome complex are listed in below Table 3. All quantitation is relative; note, however, that in this example the ratio was calculated as Test/WT. Samples were digested with trypsin and analyzed on an Orbitrap Eclipse MS system. Data were analyzed in Proteome Discoverer 2.4 using Sequest and Percolator algorithms. Values shown represent multiple consensus based quantitation. Note, that in the porosome complex from the ΔF508-CFTR cell line (the test cell sample), the Ras GTPase-activating-like protein IQGAP1 is absent. This suggests that the IQGAP1 gene product may be interacting with the CFTR protein in the porosome complex in the native state. Upregulating porosome GTPase activity may thus potentially be used as a therapy for CF. Likewise, practitioners of the teachings herein may choose porosome GTPase as a porosome target protein acted upon by either a humanized nanobody, a small molecule, or both.

TABLE 3
MASS SPECTROMETRY ON POROSOME ISOLATED FROM WT-CFTR
HUMAN BRONCHIAL EPITHELIAL CELL LINE AND EXPERIMENTAL
ΔF508-CFTR CELL LINE ΔF508del (−/−)

| | | | | | | | Fold |
|---|---|---|---|---|---|---|---|
| | | | Abundance | WT | Abundance | Test | Change |
| Protein Name | Gene | p-value | WT Mean | Stdev | Test Mean | Stdev | Test/WT |
| Ras GTPase-activating- | IQGAP1 | NA | 1.50E+06 | 8.64E+05 | | | |
| like protein IQGAP1 OS | | | | | | | |
| Myosin-9 OS | MYH9 | 0.006194 | 8.71E+07 | 3.66E+07 | 8.58E+06 | 4.63E+06 | 0.10 |
| Myosin light polypeptide | MYL6 | NA | 3.34E+06 | 8.49E+05 | 3.57E+05 | 9.97E+04 | 0.11 |
| 6 OS | | | | | | | |
| Calmodulin-like protein | CALML3 | NA | 3.13E+06 | NA | 5.14E+05 | | 0.16 |
| 3 OS | | | | | | | |
| Proton-activated chloride | PACC1 | 0.18244 | 3.04E+06 | 1.69E+06 | 9.20E+05 | 5.05E+05 | 0.30 |
| channel OS | | | | | | | |
| Transgelin-2 OS | TAGLN2 | 0.00816 | 1.52E+06 | 6.09E+05 | 5.20E+05 | 1.66E+05 | 0.34 |
| Moesin OS | MSN | 0.043244 | 5.80E+05 | 1.27E+04 | 3.54E+05 | 1.12E+05 | 0.61 |
| Cofilin-1 OS | CFL1 | 0.032227 | 1.07E+07 | 2.45E+06 | 8.05E+06 | 1.47E+06 | 0.75 |
| Tubulin beta chain OS | TUBB | 0.017152 | 2.12E+07 | 1.20E+07 | 2.01E+07 | 3.45E+06 | 0.95 |
| Ezrin OS | EZR | 0.064013 | 2.01E+06 | 2.51E+05 | 2.27E+06 | 9.05E+05 | 1.13 |
| Actin, cytoplasmic 1 OS | ACTB | 0.00117 | 1.31E+08 | 1.06E+07 | 1.53E+08 | 2.62E+07 | 1.17 |
| Reticulon-4 OS | RTN4 | 0.00894 | 2.43E+05 | 2.30E+04 | 2.93E+05 | 3.95E+04 | 1.21 |
| Voltage-dependent | VDAC3 | 0.036505 | 1.04E+06 | 1.11E+06 | 1.68E+06 | 8.73E+05 | 1.62 |
| anion-selective channel | | | | | | | |
| protein 3 OS | | | | | | | |
| Synaptosomal-associated | SNAP23 | 0.00061 | 6.38E+08 | 8.64E+07 | 1.06E+09 | 4.10E+07 | 1.66 |
| protein 23 OS | | | | | | | |
| ADP-ribosylation factor | ARF3 | 0.206311 | 3.08E+06 | 3.05E+06 | 5.53E+06 | 2.89E+06 | 1.80 |
| 3 OS | | | | | | | |
| Ras-related protein Rab- | RAB3C | 3.97E−05 | 7.65E+05 | 1.47E+05 | 1.41E+06 | 2.06E+05 | 1.85 |
| 3C OS | | | | | | | |
| Ras-related protein Rab- | RAB1A | 0.050062 | 2.15E+06 | 4.07E+05 | 4.83E+06 | 1.21E+06 | 2.25 |
| 1A OS | | | | | | | |
| Ras-related protein Rab- | RAB1B | 0.050062 | 2.15E+06 | 4.07E+05 | 4.83E+06 | 1.21E+06 | 2.25 |
| 1B OS | | | | | | | |
| Filamin-A OS | FLNA | 0.000489 | 1.33E+06 | 2.77E+05 | 4.08E+06 | 3.29E+05 | 3.06 |
| Voltage-dependent | VDAC1 | 0.165242 | 1.16E+06 | 1.11E+06 | 4.38E+06 | 3.60E+06 | 3.78 |
| anion-selective channel | | | | | | | |
| protein 1 OS | | | | | | | |
| Vimentin OS | VIM | NA | | | | | |
| Spectrin beta chain, non- | SPTBN5 | NA | | | 2.78E+06 | | |
| erythrocytic 5 OS | | | | | | | |
| Tubulin alpha-1A chain | TUBA1A | NA | | | | | |
| OS | | | | | | | |

* Note, that in the porosome complex from the ΔF508-CFTR cell line, the Ras GTPase-activating-like protein IQGAP1 is absent, suggesting interaction with the CFTR protein within the native porosome complex.

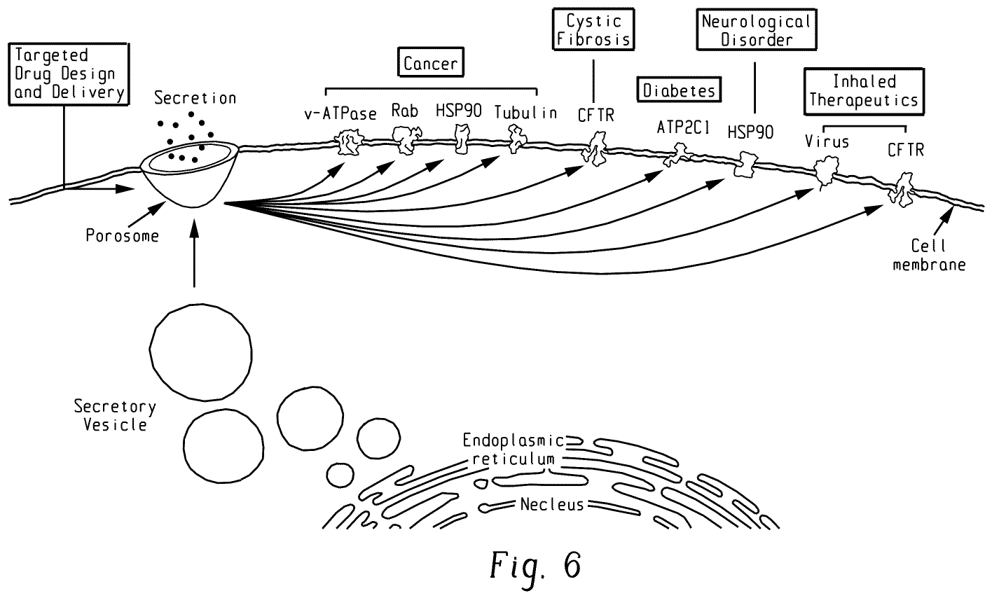

FIG. 6 is depiction of porosome proteins found at play in particular disease classes. FIG. 6 further depicts some of the breadth of diseases addressable using embodiments of the porosome-targeted tissue-specific drug design, development, and delivery platform herein detailed. defective secretory functions are addressed via the porosome. Secretory defects could be caused by a defect in one or more porosome proteins resulting in either excessive secretion as in certain cancers or due to a decrease or loss of appropriate secretions such as in diabetes or cystic fibrosis.

Excessive secretion can be treated by specific small molecules that bind and interact with one or more porosome proteins to ameliorate the defect. For example, t-SNARE or v-SNARE nanobodies could be used to modulate the decrease in secretion.

In case of an under-functional or defective porosome, the entire functional porosome could be reconstituted into the defective tissue as, for instance, with the lung epithelia in the case of cystic fibrosis. In some embodiments, for the reconstitution, a porosome from pig or human sources is extracted and put in a human cell. In still other embodiments, the nanoscale porosome complex for reconstitution is obtained from CALU 3 or other human airway epithelial cells to address cystic fibrosis. Those skilled in the art, upon reviewing the porosome proteins and their role in diseases as at least disclosed herein can readily envision additional cell types suitable for reconstitution. Therefore, reconstitution therapy involves reconstituting or introducing a normal functional CFTR-associated secretory porosome complex—the entire 100 nanometer porosome complex containing the CFTR protein—at the cell plasma membrane of the lung epithelia in CF patients. Reconstitution addresses the different CFTR mutation issues. Reconstitution therapy will ameliorate defects in mucus secretion caused by the mutated malfunctioning CFTR.

Further below detailed in Table 4, are classes of porosome proteins and their purported role in diseases.

TABLE 4
CLASSES OF POROSOME PROTEINS AND
THEIR PURPORTED ROLE IN DISEASES

| TARGET | INDICATION |
|---|---|
| CNPase | Alzheimer's, Down syndrome & |
| | Schizophrenia |
| HSP70 | Alzheimer's |
| DRP-2 | Alzheimer's |
| SNAP-25 | Alzheimer's, Down syndrome & |
| | Schizophrenia |
| Ras/Rho GTPases | Gastric carcinoma |
| V-ATPase | NSCLC |
| Tubulin beta | Ovarian, breast and gastric cancers |
| Phosphatidylserine | Pancreatic, lung, brain, and pediatric tumors |
| Myosin 7b | Cancer, Cardiac disease |
| Creatine Kinase | NSCLC |
| Langerin | Langerhans cell histiocytosis |
| Cdc-42-Intersectin | Cancers, tumors |
| Dihydropyrimidinase-like | NSCLC |
| proteins (DPYSLs) | |
| CFTR Chloride Channel | Cystic fibrosis |

### Neuronal Porosome Proteins

In Alzheimer's, as seen in Table 4, the proteins 2,3-cyclic nucleotide phosphodiesterase (CNPase) and the heat shock protein 70 (HSP70) are implicated as playing a role in disease pathology. The levels of CNPase and HSP70, both present in the neuronal porosome complex are found to increase, while the levels of porosome-associated dihydropyrimidinase-related protein-2 (DRP-2) is decreased. Similarly, porosome proteins SNAP-25 and synaptophysin are significantly reduced in neurons of patients with Alzheimer's disease.

Decreased levels of CNPase have also been reported in the frontal and temporal cortex of patients with Alzheimer's disease and Down syndrome. Low CNPase levels have also been detected in the anterior frontal cortex in schizophrenic patients. Additionally, an allele that is associated with low levels of CNPase is also reported to be linked to Schizophrenia.

Examples of neuronal porosome proteins can include: Tubulin beta, myosin 7b, spectrin, Creatine kinase, Dystrophin, Langerin, GTPase activating protein (GAP), Intersectin 1 isoform (ITSN-1), Actin, cytoplasmic 1, Sodium/potassium-transporting ATPase subunit alpha-3, Plasma membrane calcium-transporting ATPase 1, Plasma membrane calcium-transporting ATPase 2, Brain acid soluble protein 1, Adenylyl cyclase-associated protein 1, 2′, 3′-Cyclic-nucleotide 3′-phosphodiesterase, Dihydropyrimidinase-related protein 2, Dihydropyrimidinase-related protein 3, Dihydropyrimidinase-related protein 5, Glutamine synthetase, Guanine nucleotide-binding protein G(o) subunit alpha, Neural cell adhesion molecule 1, Vesicle-fusing ATPase, Ras-related protein Rab-3A, Reticulon-3, Reticulon-4, Synaptosomal-associated protein 25, Syntaxin-1A, Syntaxin-1B, Syntaxin-binding protein 1, Synapsin-2, Synaptophysin, Synaptotagmin-1, Tubulin alpha-1A chain, Vesicle-associated membrane protein 1, Vesicle-associated membrane protein 2, V-type proton ATPase subunit B, brain isoform. Embodiments of the invention can include one or more identified small molecules that directly act upon one or more of the above proteins to affect neuronal porosome structure and/or function.

### Insulin Secreting Porosome Proteins—Diabetes

According to the Centers for Disease Control and Prevention (CDC), more than 37 million Americans have diabetes (1 in 10), and approximately 90-95% of them have type 2 diabetes. Although type 2 diabetes typically develops in people over 45 years of age, there is a growing number of children developing it. The hormone insulin is made and secreted in β-cells of the endocrine pancreas. Insulin acts on cells in the body to let sugar in for use as energy. In type 2 diabetes, cells don't respond to insulin, a condition called insulin resistance. Consequently, the β-cells produce more insulin to try to get cells to respond. Eventually the β-cells cannot elevate production to keep up, and blood sugar rises, setting the stage for type 2 diabetes. Elevated blood sugar is damaging to the physiology and can cause serious health problems such as cardiovascular diseases, blindness, kidney disease and dementia. Type 2 diabetes can be managed by healthy diet and medication, but these alone are often not wholly successful with current medications often resulting in undesired side-effects.

Following a meal, secretion of digestive enzymes from the exocrine pancreas aids in food digestion. The consequent elevation of blood glucose following digestion triggers the secretion of insulin from the β-cells of the endocrine pancreas. Glucose-stimulated release of insulin stored in secretory vesicles in β-cells occurs either by complete collapse of the vesicle membrane at the cell plasma membrane, or the transient fusion of secretory vesicles at the base of plasma membrane-associated porosomes. In some embodiments of the invention, the functional reconstitution of the insulin-secreting porosome complex in live β-cells of the endocrine pancreas, opens a window in the treatment of diabetes.

Therefore, in type 2 diabetes there are primarily two problems: one, the β-cells in the pancreas do not produce enough insulin, and two, cells in the body respond poorly to insulin to let sugar in. Currently, there is no cure for type 2 diabetes. It can only be managed through diet, physical activity, combined with the following available medications:

Metformin (Fortamet®, Glumetza®, others) is generally the first medicine prescribed for type 2 diabetes. It works mainly by lowering glucose production in the liver and improving the sensitivity of the body to insulin, so it uses insulin more effectively. Metformin is known to inhibit insulin secretion from β-cells of the endocrine pancreas. Patients taking Metformin, experience B-12 deficiency and may need to take supplements. Other possible side effects of Metformin include nausea, abdominal pain, diarrhea and bloating.

Metformin is also sometimes combined with other medications. Among them, Sulfonylureas, Glinides, Thiazolidinediones, DPP-4 inhibitors, GLP-1 receptor agonists, SGLT2 inhibitors, and insulin therapy.

Sulfonylureas help the body secrete more insulin. Examples include glyburide (DiaBeta®, Glynase®), glipizide (Glucotrol XL®) and glimepiride (Amaryl®). Possible side effects include low blood sugar and weight gain.

Glinides stimulate the pancreas to secrete more insulin. They're faster acting than sulfonylureas, however their effect in the body is shorter. Examples include repaglinide and nateglinide. Possible side effects include low blood sugar and weight gain.

Thiazolidinediones make the body's tissues more sensitive to insulin. An example of this medicine is pioglitazone (Actos®). Possible side effects include risk of congestive heart failure, risk of bladder cancer (pioglitazone), risk of bone fractures, and weight gain.

DPP-4 inhibitors help reduce blood sugar levels but tend to have a very modest effect. Examples include sitagliptin (Januvia®), saxagliptin (Onglyza®) and linagliptin (Tradjenta®). Possible side effects include risk of pancreatitis and joint pain.

GLP-1 receptor agonists are injectable medications that slow digestion and help lower blood sugar levels. Their use is often associated with weight loss, and some may reduce the risk of heart attack and stroke. Examples include exenatide (Byetta®, Bydureon Bcise®), liraglutide (Saxenda®, Victoza®) and semaglutide (Rybelsus®, Ozempic®, Wegovy®). Possible side effects include risk of pancreatitis, nausea, vomiting and diarrhea.

SGLT2 inhibitors affect the blood-filtering functions in the kidneys by blocking the return of glucose to the bloodstream. As a result, glucose is removed in the urine. These medicines may reduce the risk of heart attack and stroke in people with a high risk of those conditions. Examples include canagliflozin (Invokana®), dapagliflozin (Farxiga®) and empagliflozin (Jardiance®). Possible side effects include vaginal yeast infection, urinary tract infection, low blood pressure, high cholesterol, risk of gangrene, risk of bone fracture (canagliflozin) and risk of amputation (canagliflozin).

Some people who have type 2 diabetes need insulin therapy. In the past, insulin therapy was used as a last resort, but today it may be prescribed sooner if blood sugar targets are missed despite lifestyle changes and other medicines. Different types of insulin vary on how quickly they begin to work and how long they have an effect. Long-acting insulin, for example, is designed to work overnight or throughout the day to keep blood sugar levels stable. Short-acting insulin generally is used at mealtime to moderate any accompanying spikes in blood sugar associated with a meal.

In summary, the current available medications to manage type 2 diabetes follow the strategies of: lowering glucose production; improving the sensitivity of the body to insulin; increasing insulin secretion; and increasing excretion of glucose by kidney. Currently, there are no drugs that increase both the production and secretion of insulin by β-cells of the endocrine pancreas.

Examples of insulin secreting porosome proteins can include: Actin, cytoplasmic 1, Tubulin alpha-1A, Cofilin-1, Calcium-transporting ATPase type 2C, Ankyrin repeat-domain containing protein, Unconventional myosin-X, Rab11 family-interacting protein 4, Arf-GAP with SH3 domain, Transmembrane protein 194A, Rab4, 6, 33, 10, 15, 35, 1, 38, 27, 39, Rab GDP dissociation inhibitor alpha, Potassium channel subfamily K member 2, Rho GTPase-activating protein 40, Heat shock protein HSP 90, Heat shock cognate 71 kDa protein, Synaptosomal-associated protein 25, Ankyrin repeat-domain containing protein, Profilin-1, Tubulin beta 2A, Destrin, Guanine nucleotide binding protein beta-2, Rho GDP-dissociation inhibitor 1, Calmodulin, Microtubule associated protein 1 and 2, ADP-ribosylation factor 5, ADP-ribosylation factor-like protein 3, Apolipoprotein A-1, Arf-GAP. Embodiments of the invention can include one or more identified small molecules that directly act upon one or more of the above proteins to affect insulin secreting porosome structure and/or function. In still other embodiments, a small molecule may enhance the ability of a porosome constituent protein to form a porosome assembly.

In some embodiments, a small molecule, such as 17-demethoxy-17(2-prophenylamino) geldanamycin, may regulate a heat shock protein, such as HSP90, to affect porosome structure formation and subsequent function. In some embodiments, a small molecule may disrupt the formation of a porosome structure through the inhibition of component protein binding and assembly.

Additional embodiments of the invention comprise a porosome protein that regulates both the expression and secretion of insulin in β-cells of the endocrine pancreas. The identified insulin secreting porosome proteins are ATP2C1 (ATPase secretory pathway Ca+2 transporting 1) and APOa1, that shows both the expression and secretion of insulin in β-cells of the endocrine pancreas. These proteins were identified using CRISPR knock out and the over expression of different insulin-secreting porosome proteins. The above identified proteins belong to the family of P-type cation transport ATPases. This magnesium-dependent enzyme catalyzes the hydrolysis of ATP coupled with the transport of calcium ions.

In still another embodiment of the invention there is a method for identifying one or more modulator(s) of the insulin-secreting porosome protein(s), to modulate insulin production and secretion from β-cells of the endocrine pancreas. Certain embodiments employ the mRNA induced over expression of ATP2C1 and APOa1 in β-cells. Similarly, CDN1163, a Ca2+-ATPase (SERCA) activator is identified to be able to be used in increasing both the expression and secretion of insulin in β-cells of the endocrine pancreas. This activator, in some embodiments may be used alone or in combination with modulators.

The Ca2+-ATPase (SERCA) activator CDN1163, is identified to be able to be used in increasing both the expression and secretion of insulin in β-cells of the endocrine pancreas, alone or in combination with Metformin and or other existing above-mentioned type 2 diabetes drugs, including modulators of the ATP2C1-associated porosome proteins within the secretory complex.

Certain embodiments of the invention use the above-described method for identifying protein-protein interactions within functional complexes in cells. In particular the protein-protein interactions within the insulin-secreting porosome complex in β-cells of the endocrine pancreas enable the fine tuning of the regulation of the insulin-secreting porosome secretory machinery in β-cells of the endocrine pancreas and its precision targeting using small molecule drug-nanobody complexes.

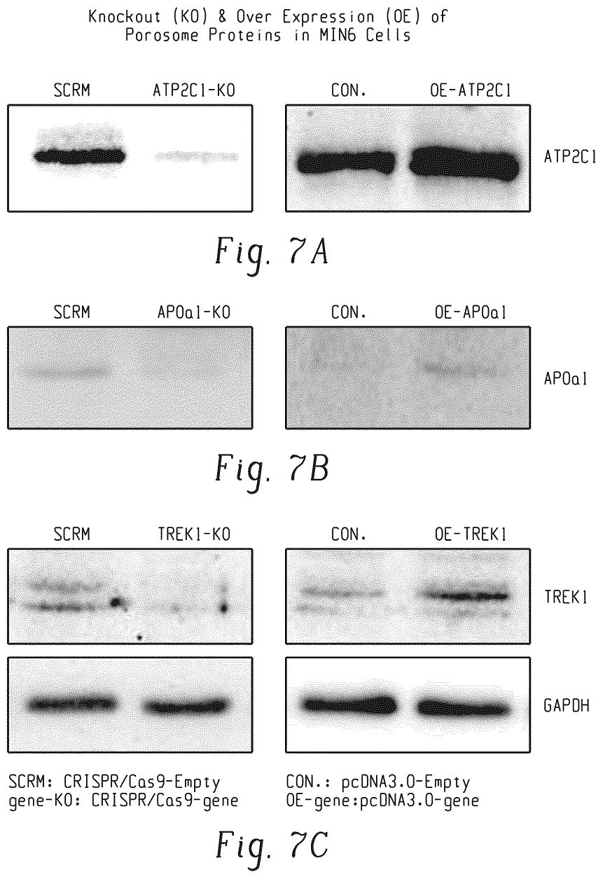

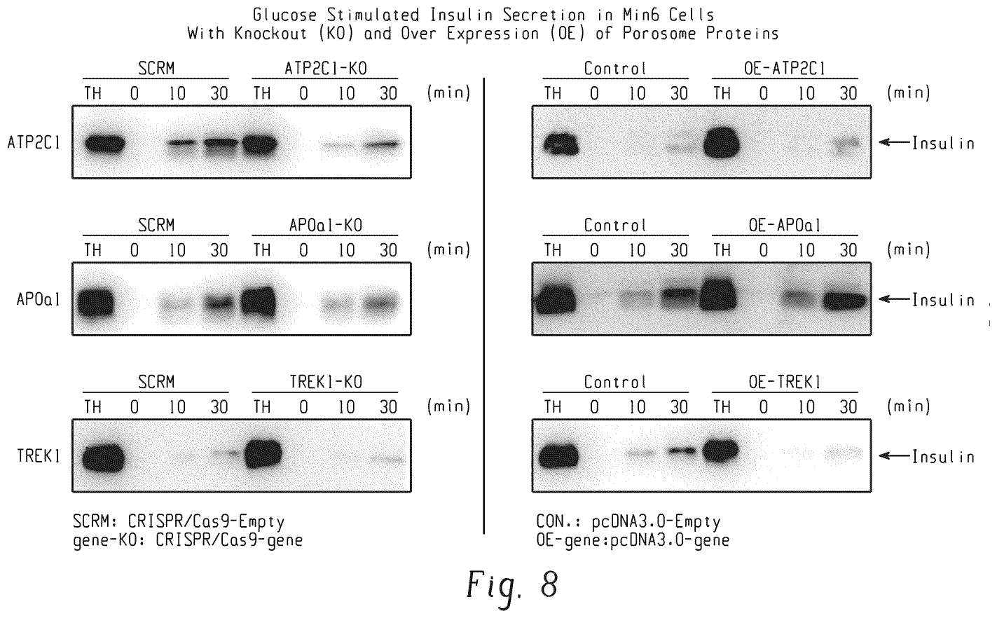

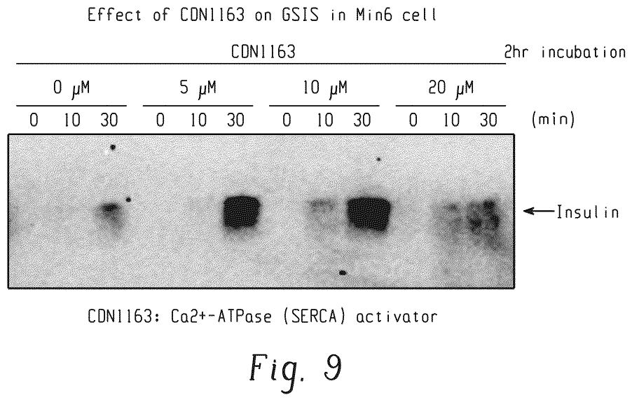

As shown in FIG. 7, in still other embodiments, over expression of different insulin-secreting porosome proteins enabled the identification of insulin secreting porosome proteins ATP2C1 (ATPase secretory pathway Ca+2 transporting 1) and APOa1, that showed both the increased expression and glucose-stimulated secretion of insulin in β-cells of the endocrine pancreas (as shown in FIG. 8). Hence, the Ca2+-ATPase activator CDN1163, can be used to increase both the expression of insulin and the glucose-stimulated insulin release (see FIG. 9) from β-cells of the endocrine pancreas. Indeed, FIG. 7 illustrates the knockout (KO) and over expression (OE) of three porosome proteins: ATP2C1, APOa1 and TREK1 in Min6 cells. Note the Western blots on the CRISPR/Cas9 empty (SCRM) and KO and the control (CON) and over expressed proteins. GAPDH are loading controls. For its part, FIG. 8 illustrates the glucose-stimulated insulin secretion in knockout (KO) and over expression (OE) of the three porosome proteins: ATP2C1, APOa1 and TREK1 in Min6 cells. Note that there is significant loss of insulin secretion at the 10 min and 30 min time points in the ATP2C1 KO compared to the other two KO's (i.e., APOa1 and TREK1). Interestingly, the OE of ATP2C1 and APOa1 results in both the increased synthesis and secretion of insulin, while TREK1 exhibits a decrease in both synthesis and secretion of insulin. Finally, as seen in FIG. 9, glucose-stimulated insulin secretion in Min6 cells following a 2 h exposure to the ATP2C1 activator CDN1163 is increased. Note that 10 uM CDN1163 is optimal in glucose-stimulated insulin secretion.

These results demonstrate that targeted overexpression of ATP2C1 or APOa1 or both, in β-cells of the endocrine pancreas using mRNA or other gene therapies would be a treatment of type 2 diabetes. Similarly, small molecule activators of both porosome proteins can serve as drugs in the treatment of type 2 diabetes.

### Porosome Reconstitution Therapy for Type 1 Diabetes (T1D)

Current T1D diabetes therapy is based on insulin injections and cadaveric islets transplantation, which has many disadvantages. Consequently, new methods are being developed to regenerate the pancreatic hormone-producing cells in vitro. The most promising approach is the generation of stem cell (SC)-derived β-cells that could provide an unlimited source of insulin. Recent studies provide methods to produce β-cell-like cell clusters that display glucose-stimulated insulin secretion, one of the key characteristics of the β-cell. However, compared to native β-cells, SC-derived β-cells do not undergo full functional maturation; and, hence, exhibit limited glucose-stimulated insulin secretion creating a need that urgently needs to be addressed. Results from ongoing clinical trials indicates that the existing protocols to generate SC-islets capable of improving glycemic control in human T1D patients require further enhancement of insulin secretion that mimics primary adult islets. This would reduce the number of cells required for transplantation and make it easier to manufacture sufficient cell numbers for therapy. It is reported that, if insulin secretion per cell is doubled, then potentially only half as many cells would be required to cure a patient. Reducing graft volume would additionally facilitate an easier transplantation procedure, reduce nutrient exchange requirements at the transplantation site, and provide the possibility for alternate transplantation sites. With the requirement of fewer cells for a successful transplant, there would be a significant decrease in the requirement of both production costs and logistics for a cell-based therapy. Insulin-secreting porosome-reconstitution into SC-Islet will enable the enhancement of glucose-stimulated insulin secretion and solve this problem.

### Exosome Release Control

In addition to secretion of neurotransmitters, digestive enzymes or hormones, cells also communicate with each other via secreted membrane-bounded nanostructured extracellular vesicles called exosomes, first discovered and reported in 1983 as a selective externalization mechanism of the transferrin receptor in sheep reticulocytes. Electron micrographic evidence for such externalization of the transferrin receptor in vesicular form from sheep reticulocytes, was demonstrated in 1985. In the past 35 years, great progress has been made in our understanding of the biology, function, and biomedical application of exosomes. Exosome vesicles are packaged with proteins, DNA, and RNA, that are destined for specific target cells in the body. Such intercellular communication via exosomes has also been implicated in various pathologies such as cancers, neurological disorders and inflammatory disease. While extracellular vesicle cargo includes plasma membrane and endosomal proteins, they may also contain materials from various intracellular compartments such as the mitochondria. Studies report the presence of mitochondrial DNA within extracellular vesicles. Although, multi-vesicular bodies may fuse at the cell plasma membrane to release their cargo, the molecular mechanism of exosome release and or their cargo from various cell types, remains unclear. It is believed that exosome release may occur via the porosome complex.

In some embodiments, exosome release is controlled via the altered structure and/or functioning of the porosome complex or porosome-associated proteins. In certain embodiments, one or more cell types may have genes of putative porosome complex or porosome-associated proteins “knocked out” through the use of CRISPER, RNAi, or other methods such as are known in the art.

For example, in the Min6 cells of rat brains a knockout of ATP2C1 was generated by genome editing using a CRISPER/Cas9 system. Comparison of such cells to control wild type (SCRM) cells illustrated the release of exosomes via the porosome complex, demonstrating a loss of both glucose-stimulated insulin secretion. Thus, small molecules that enhance or alter the production of ATP2C1 likely have an effect on insulin secretion.

### Nanobodies

Nanobodies are a subclass of antibodies composed of a single polypeptide chain with versatile molecular binding scaffolds found in the camel species, as opposed to the large y-shaped conventional antibodies found in other mammalian species including humans. In certain embodiments of the invention engineered nanobodies against different porosome protein targets are used to bind the nanobody to the protein target and thus precision-target drugs to specific porosome proteins and or alter the structure and function of the porosome. In some embodiments variable domains of the camelidae variable domain (VHH) nanobodies are humanized to target and bind to one or more domains of one or more porosome proteins. Nanobodies targeted to multiple porosome proteins besides helping in precision targeting of small molecules may be used to physically or chemically alter the structure and/or function of the porosome, thus altering the course of a porosome-mediated disease. Thus, one or more nanobodies of the same or different classes may be linked to one or more small molecules to fine tune targeting to, and response of, the porosome structure-function outcome.

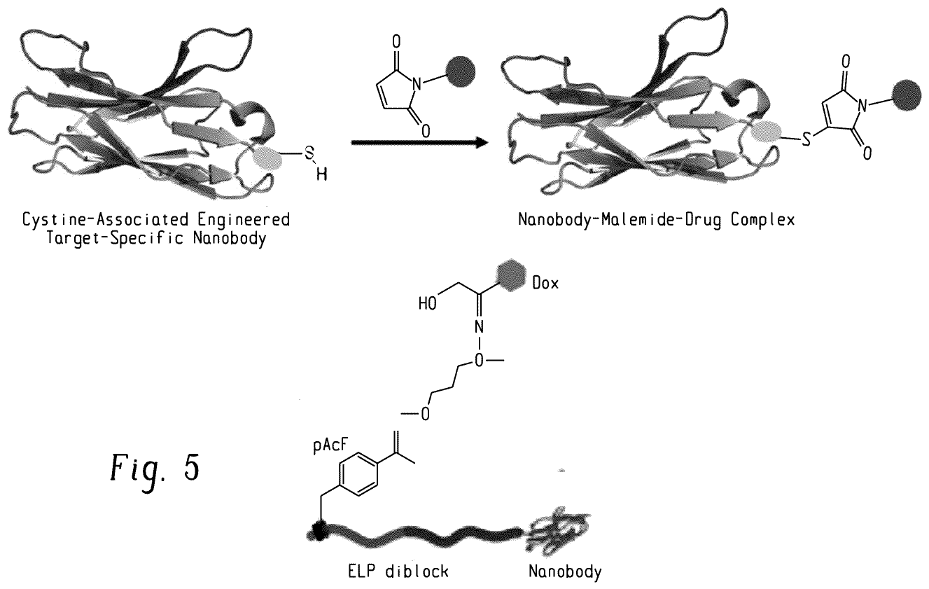

FIG. 5 illustrates drug targeting specificity using a combinatorial small molecule drug design combined with tissue-specific and porosome-specific multivalent nanobodies (top). Further illustrated are site-specific functionalization of nanobody can be achieved via engineered cysteine (centered, bottom). Cysteine is introduced into nanobody through genetic modification. Maleimide is one of the most widely used sulfhydryl-reactive chemical groups. Yellow oval indicates backbone of foreign cysteine. Red sphere denotes functional group (small molecule drug) attached to maleimide. Finally, there is provided an example of the chemistry involved in such a preparation (Bottom): the incorporation of the amino acid p-acetyl phenylalanine (pAcF), provides a biorthogonal ketone for the attachment of the cancer drug doxorubicin (Dox) in the presence of reactive amino acids in a nanobody-targeted, elastin-like polypeptide nanoparticle (ELP diblock). Diblock copolymers based-on elastin-like polypeptide (ELP) have the potential to undergo specific phase transitions when thermally stimulated. This ability is especially suitable to form carriers, micellar structures for instance, for delivering active cargo molecules Similarly, multivalent nanobodies having both tissue-specific and porosome-specific domains can be generated for precision targeting of combinatorial small molecule drug. In this instance the term “multivalent” refers to a common operational usage of the term to illustrate more than one nanobody, or molecule, or otherwise functional element formed as part of the combinatorial drug.

### Examples of Neuronal Diseases and Cancers

As previously mentioned, the porosome structure includes multiple proteins with their associated ligands, chaperones, and other affiliated molecules such as lipids. Although it is classically understood that there are diseases caused by mutations/malformations in the structure of a single protein, as discussed supra, it is only recently starting to be understood that malfunction and malformations of larger structures, such as the porosome, contribute to diseases. The following non-limiting examples illustrate examples of porosome protein malfunctions and their contribution to a disease followed by example single-target small molecules that may affect the disease states.

Neuronal Diseases: (e.g., Alzheimer's, Down syndrome & Schizophrenia): In Alzheimer's, the levels of the porosome protein CNPase (2,3-cyclic nucleotide phosphodiesterase) and the heat shock protein 70 (HSP70), both present in the neuronal porosome complex are found to increase, while the levels of porosome-associated dihydropyrimidinase-related protein-2 (DRP-2) is decreased. Similarly, porosome proteins SNAP-25 and synaptophysin are significantly reduced in neurons of patients with Alzheimer's disease. Similarly, in Down syndrome & Schizophrenia: Decreased levels of the porosome protein CNPase have also been reported in the frontal and temporal cortex of patients with Alzheimer's disease and Down syndrome. Low CNPase levels have also been detected in the anterior frontal cortex in schizophrenic patients. Additionally, an allele that is associated with low levels of CNPase is also reported to be linked to Schizophrenia.

Small molecule inhibitors & stimulators of porosome phosphodiesterase such as Vinpocetine, BAY 60-7550, Rolipram, Etazolate, Sildenafil, S14, VP1.15, PF-04447943, Papaverine, and the small molecule inhibitors Apoptozole, VER155008, JG98, HA15 and YUM70 of HSP70, and small molecule activator ML346 for HSP70, all may be used to treat neuronal diseases especially Alzheimer's.

Cancers: Cellular secretion is an important mediator of cancer progression. For example, the Ras superfamily of small GTPases present in porosomes are implicated in 33% of human cancers. However, direct pharmacological inhibition of Ras mutants remains challenging. Therefore, the alternate strategy is to inhibit porosome protein V-ATPase activity while continuing to screen and design new and novel small molecules that directly bind and inhibit Ras GTPases. Similarly, non-small cell lung cancer (NSCLC) has a dismal prognosis and remains the most common cause of cancer-related death worldwide. The porosome protein tubulin beta is strongly associated with drug-refractory and aggressive NSCLC. βIII-tubulin is also associated with resistance to taxanes or vinorelbine in a range of tumor types, including ovarian, breast and gastric cancers. Small molecule drugs targeting microtubules such as Docetaxel, Taxol, Podophyllotoxin, Etoposide, Vinblastine, Vincristine, Vinorelbine, Griseofulvin, Cytocholasin A and E, TN-16, Myoseverin, Nocodazole, Vindesine, Phomopsin A, d-24851, Monastrol, AMP-PNP, Adociasulfate-2, Terpendole-E, Tubacin, Scriptaid, DPD, and C2-8, may be used in the combinatorial approach above outlined.

Small molecules targeting porosome lipids involved in cancer therapy may also serve in embodiments of the invention. Studies report that cholesterol at the cell plasma membrane is critical for secretion from cells. Cholesterol depletion from the cell membrane reduces the incorporation of phosphatidylserine (PS) from entering the cell plasma membrane, resulting in loss of secretion. Phosphatidylserine (PS) is normally located in the inner leaflet of the membrane bilayer of healthy cells, however it is expressed at high levels on the surface of cancer cells. This has allowed for the development of selective therapeutic agents against cancer cells (without affecting healthy cells). For example, SapC-DOPS is a PS-targeting nanovesicle which effectively targets and kills several cancer types including pancreatic, lung, brain, and pediatric tumors. SapC-DOPS selectively induces apoptotic cell death in malignant and metastatic cells, whereas untransformed cells remain unaffected due to low surface PS expression. In still another approach, plasma membrane cholesterol-depleting small molecule such as cyclodextrins in combination with SapC-DOPS, are potentially useful for cancer therapy.

Table 5 presents additional porosome proteins and small molecule drugs targeting porosome proteins & lipids while, when possible, explaining their potential role in treating various diseases.

TABLE 5
ADDITIONAL POROSOME PROTEINS AND SMALL MOLECULE
DRUGS TARGETING POROSOME PROTEINS

| | Small Molecule Drugs Targeting Porosome Proteins & Lipids |
|---|---|
| Porosome Proteins | (Role in treating various diseases including cancer) |
| Ras/Rho GTPases | Mutations in Ras family proteins are implicated in 33% of human |
| | cancers, but direct pharmacological inhibition of Ras mutants |
| | remains challenging. The Ras superfamily of small GTPases is |
| | essential for a wide range of cellular processes including secretion. |
| | Mutations or dysregulation of these proteins are associated with |
| | various diseases including cancer. However, the unsuccessful |
| | attempt to directly target these small GTPases has resulted in their |
| | classification as “undruggable”. Recent studies report several small |
| | molecules which may work. An alternate strategy is to inhibit the |
| | activity of the porosome protein V-ATPase |
| Tubulin beta | Alpha and beta tubulin polymerize to form microtubules. |
| | Example small molecule drugs targeting microtubules: Docetaxel, |
| | Taxol, Podophyllotoxin, Etoposide, Vinblastine, Vincristine, |
| | Vinorelbine, Griseofulvin, Cytocholasin A and E, TN-16, |
| | Myoseverin, Nocodazole, Vindesine, Phomopsin A, d-24851, |
| | Monastrol, AMP-PNP, Adociasulfate-2, Terpendole-E, Tubacin, |
| | Scriptaid, DPD, C2-8. |
| Myosin 7b | Myosin heavy chain 7b is an ancient member of the myosin heavy |
| | chain motor protein family and a regulatory long noncoding RNA |
| | derived protein. |
| | Example small molecule myosin inhibitors: Blebbistatin, N- |
| | benzyl-p-toluene sulphonamide (BTS), 2,3-Butanedione monoxime |
| | (BDM), Pentachloropseudilin (PCIP), Pentabromopseudilin |
| | (PBP), MyoVin-1, 2,4,6-Triiodophenol (TIP). |
| Creatine Kinase | The use of tyrosine kinase inhibitors (TKIs) is associated with |
| | incident creatine kinase (CK) elevation in the treatment of |
| | advanced non-small cell lung cancer (NSCLC) patients. |
| | Small molecule tyrosinr kinase inhibitors: Osimertinib, Erlotinib, |
| | Gefitinib, Icotinib, Crizotinib, Bevacizumab, Afatinib, Ceritinib, |
| | Brigatinib, Apatinib, Ensartinib, Anlotinib, Alectinib, Cetuximab, |
| | Vemurafenib. |
| Langerin | Langerin is a myeloid-C type lectin receptor. Glycomimetic small |
| | molecule ligands for the Langerin, has potential therapeutic |
| | applications in vaccine research and anti-infective therapy. |
| Intersectin 1 | Intersectin 1 is a cytoplasmic membrane-associated protein that |
| | indirectly coordinates endocytic membrane traffic with the actin |
| | assembly machinery. In addition, the intersectin 1 protein may |
| | regulate the formation of clathrin-coated vesicles and could be |
| | involved in synaptic vesicle recycling. This protein has been shown |
| | to interact with dynamin, CDC42, SNAP23, SNAP25, SPIN90, |
| | EPS15, EPN1, EPN2, and STN2. |
| | Small molecule inhibitor of Cdc-42-Intersectin: ZCL278 |
| Sodium-Potassium | Small molecule inhibitor of NKA: Ouabain, Digoxin |
| Transporting ATPase | |
| (NKA) | |
| Calcium transporting | Small molecule stimulator of CTA: CDN1163 |
| ATPase (CTA) | |
| Phosphodiesterase | Use of Phosphodiesterase Inhibitors may be found in therapy, |
| (PDE) | neuroprotection and repair. |
| | Example small molecule inhibitors of PDE: Vinpocetine, BAY 60- |
| | 7550, Rolipram, Etazolate, Sildenafil, S14, VP1.15, PF-04447943, |
| | Papaverine. |
| Dihydropyrimidinase- | DPYSLs are a family of proteins developmentally regulated |
| like proteins (DPYSLs) | during maturation of the nervous system. Members of the DPYSL |
| | family have been reported to be involved in cancer with low |
| | expression of DPYSL1 correlating with poor clinical outcomes in |
| | non-small cell lung cancer and functioning as a metastasis |
| | suppressor. |
| | Small molecule ligand: DB11638. |
| NSF or Vesicle-fusing | A small-molecule competitive inhibitor (IPA) of phosphatidic acid |
| ATPase | binding by the AAA+ protein NSF/Sec18 blocks the SNARE- |
| | priming stage of fusion. |
| | Small molecule NSF inhibitor: IPA. |
| V-type proton ATPase | Inhibition of V-type proton ATPase activity prevents secretory |
| | vesicle and lysosomal acidification, decreasing secretion and |
| | autophagy and micropinocytosis pathways that several Ras-driven |
| | cancers rely on for survival. Mutations in Ras family proteins are |
| | implicated in 33% of human cancers, but direct pharmacological |
| | inhibition of Ras mutants remains challenging. Therefore, the |
| | alternate strategy is to inhibit the V-ATPase activity. |
| | Small molecule inhibitor of V-ATPase: Bafilomycin A1 (BafA1), |
| | 249C |
| Calcium-transporting | ATP2C1: The protein encoded by this gene belongs to the family |
| ATPase type 2C | of P-type cation transport ATPases. This magnesium-dependent |
| (ATP2C1) | enzyme catalyzes the hydrolysis of ATP coupled with the transport |
| | of calcium ions. Defects in this gene cause Hailey-Hailey disease, |
| | an autosomal dominant disorder. |
| | Example small molecule inhibitor of ATP2C1: ATP2C1 mRNA is |
| | inhibited by ciclosporin, tacrolimus and vitamin D(3). |
| | Example small molecule activator of ATP2C1: Kaempferol. |
| Heat shock protein | HSP90, a late chaperonin protein is involved in the assembly of |
| HSP 90 (HSP90) | the insulin secreting porosome complex. |
| | Example small molecule inhibitor of HSP90: 17-demethoxy-17- |
| | (2-prophenylamino) geldanamycin. |
| CFTR Chloride | Cystic fibrosis transmembrane conductance regulator (CFTR), the |
| Channel | chloride channel, is present in the porosome complex of the human |
| | airway epithelia and helps in proper hydration and secretion of |
| | mucus. |
| | Example small molecules that help correctly fold and traffic |
| | CFTR: Elexacaftor, Tezacaftor, and Ivacaftor (trade name Trikafta) |
| Cholesterol | Cholesterol at the cell plasma membrane is critical for secretion |
| | from cells. Cholesterol depletion from cell membrane reduces the |
| | incorporation of phosphatidylserine (PS) from entering the cell |
| | plasma membrane. |
| | Phosphatidylserine (PS) is normally located in the inner leaflet of |
| | the membrane bilayer of healthy cells, however it is expressed at |
| | high levels on the surface of cancer cells. This has allowed for the |
| | development of selective therapeutic agents against cancer cells |
| | (without affecting healthy cells). SapC-DOPS is a PS-targeting |
| | nanovesicle which effectively targets and kills several cancer types |
| | including pancreatic, lung, brain, and pediatric tumors. SapC- |
| | DOPS selectively induces apoptotic cell death in malignant and |
| | metastatic cells, whereas untransformed cells remain unaffected |
| | due to low surface PS expression. |
| | Example small molecules that extract membrane cholesterol: |
| | Cyclodextran (CD). |

It should be understood that a reference to a pharmaceutically acceptable salt includes the solvent addition forms, particularly solvates. Solvates contain either stoichiometric or non-stoichiometric amounts of a solvent and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Functional Mucus-Secretion Porosome Reconstitution—Cystic Fibrosis Treatment

Cystic fibrosis (CF) is a disease that causes thick, sticky mucus to build up in the lungs, digestive tract, and other areas of the body. It is one of the most common life-threatening chronic lung diseases in children and young adults. Cystic fibrosis is passed down through families and is caused by a defective gene that makes the body produce abnormally thick and sticky mucus. Abnormal mucus builds up in the breathing passages of the lungs and in the pancreas. The buildup of mucus results in life-threatening lung infections and serious digestion problems. The disease may also affect the sweat glands and the male reproductive system. Many people carry a CF gene, but do not have symptoms. This is due to the fact that a person with CF must inherit 2 defective genes, one from each parent. Some CF is more common among those of northern or central European descent. Most children with CF are diagnosed by age 2, especially as newborn screening is performed across the United States. For a small number, the disease is not detected until age 18 or older. These children often have a milder form of the disease.

There are close to 40,000 children and adults living with cystic fibrosis in the United States and an estimated 105,000 people have been diagnosed with CF across 94 countries. In people with CF, a mutation in the cystic fibrosis transmembrane conductance regulator (CFTR) gene causes the CFTR protein to become dysfunctional. When the protein is not working correctly, it is unable to help move chloride ions to the cell surface. Without the chloride to attract water to the cell surface, the mucus in various organs becomes thick and sticky. In the lung, the mucus clogs the airways and traps disease causing agents, such as bacteria, leading to infection, inflammation, respiratory failure, and other complications. For this reason, avoiding germ exposure is a top concern for people with CF.

The most common mutation in the CFTR gene is delF508, a deletion of three nucleotides that results in a loss of the amino acid phenylalanine (F) at the 508th position on the protein. This mutation accounts for two-thirds (66-70%) of CF cases worldwide and 90% of cases in the United States; however, over 1500 other mutations can produce CF. Although most people have two working copies (alleles) of the CFTR gene, only one is needed to prevent cystic fibrosis. CF develops when neither allele can produce a functional CFTR protein. Thus, CF is considered an autosomal recessive disease.

There is no known cure for cystic fibrosis. Lung infections are treated with antibiotics, which may be given intravenously, inhaled, or by mouth. Sometimes, the antibiotic azithromycin is used long term. Inhaled hypertonic saline and salbutamol may also be useful. Lung transplant may be an option if lung function continues to worsen. Airway clearance technologies such as chest physiotherapy have some short-term benefits, but long-term effects are unclear. The average life expectancy is between 42 and 50 years. Lung problems (infection, decreased capacity) are responsible for death in 80% of people with cystic fibrosis.

Since there is no known cure for cystic fibrosis, treatments for CF focus on improving breathing, preventing and treating lung infections, and thinning mucus in the lung epithelia. Treatments include medicines, therapy to clear mucus out of the lungs, and in some cases, lung transplant. Pharmacogenomics approaches have led to the development of medicines that target the underlying cause of the disorder. Since different CFTR mutations have different effects on the CFTR protein, these medicines can only be used to treat people with certain CFTR mutations.

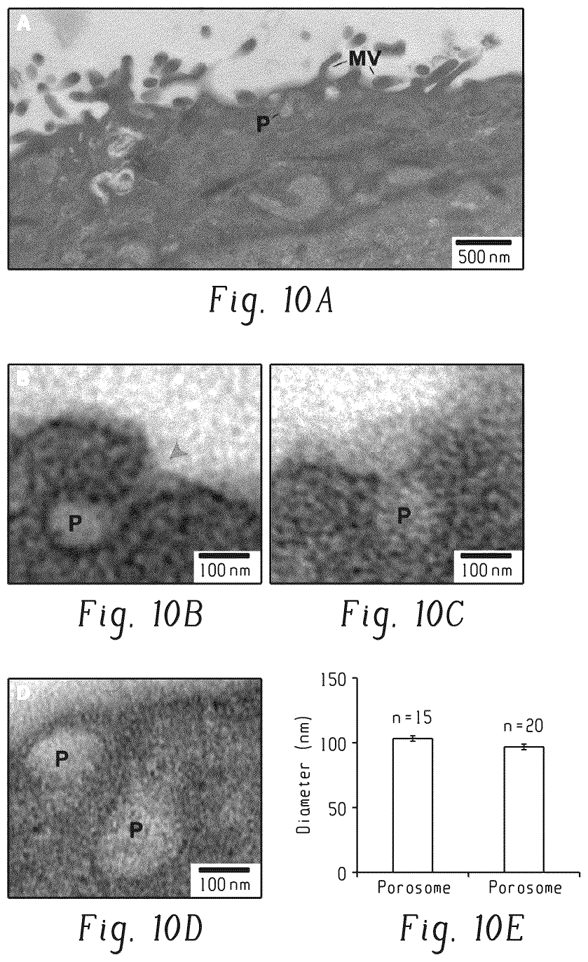

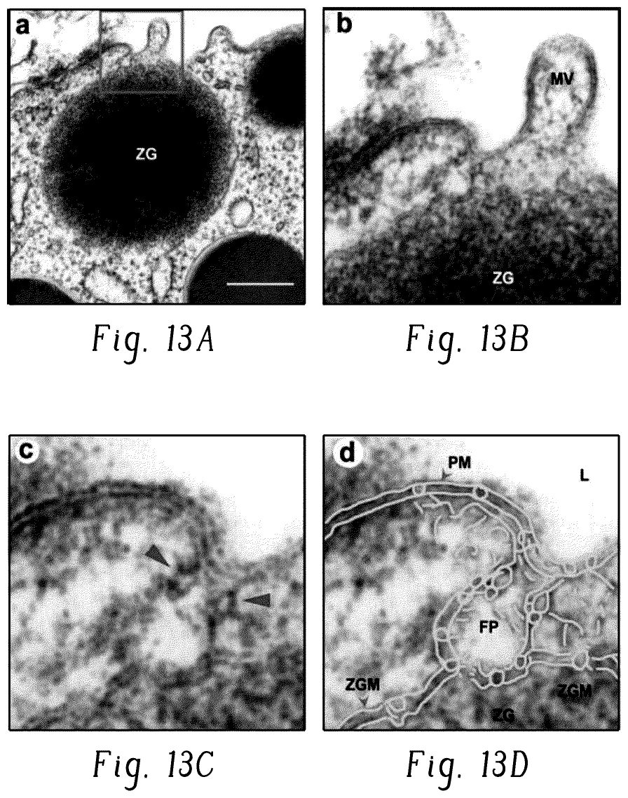

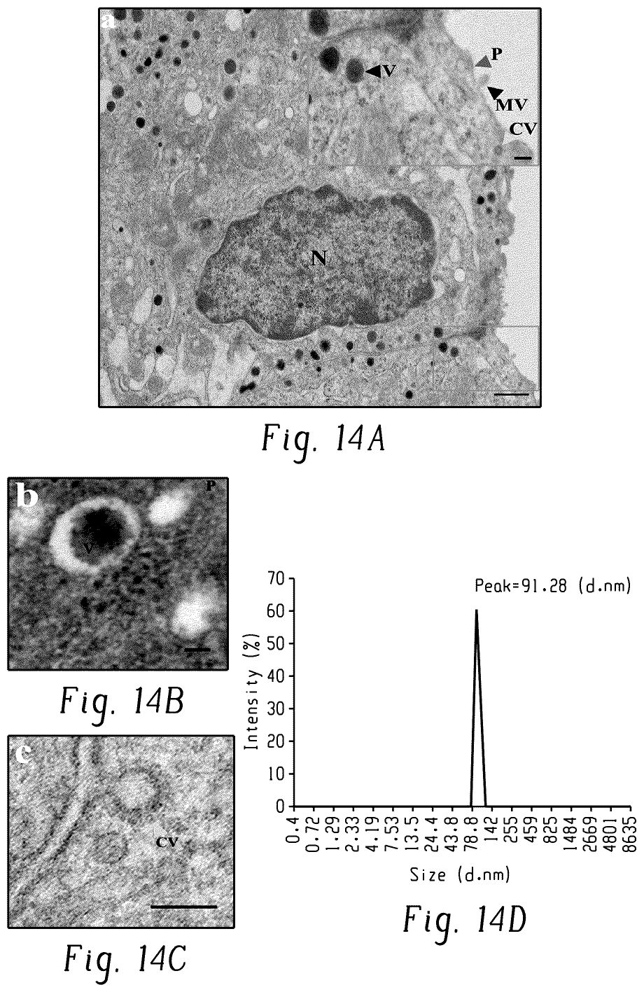

As shown in FIG. 10, the presence of porosomes in human airway epithelia is known. Likewise, the porosome proteome is known in these cell types (Table 6). Indeed, FIG. 10 presents representative electron micrographs of Calu-3 cells (cells originally obtained from a lung adenocarcinoma patient) in culture demonstrating the presence of microvilli (MV) and porosomes (P) at the cell plasma membrane. (A) Calu-3 cells demonstrate the presence of dense microvilli and porosomes at the cell plasma membrane. (B-D) Note the flask-shaped porosomes measuring nearly 100 nm in diameter (E) and from 200 to 300 nm in depth, with openings to the cell surface (red arrowhead). In (C), what appears to be mucus is found at the opening of the porosome to the cell exterior. Of the two porosomes depicted in (D), the one to the center appears to be sectioned right through the center of the organelle, whereas the porosome to the left appears to have been sectioned at its base. (E) Similar to the AFM images, the microvilli measure on average 92 nm in diameter. The human airways-associated porosome complex are similar to those present in the exocrine and endocrine pancreas shown in FIGS. 13 and 14.

Indeed, FIG. 11 illustrates the use of Us sing chamber experiments showing forskolin-stimulated chloride release from Calu-3 cells which is inhibited in the presence of the CFTR inhibitor GlyH-101. Note that the two separate experiments demonstrate similar stimulation and inhibition profiles.

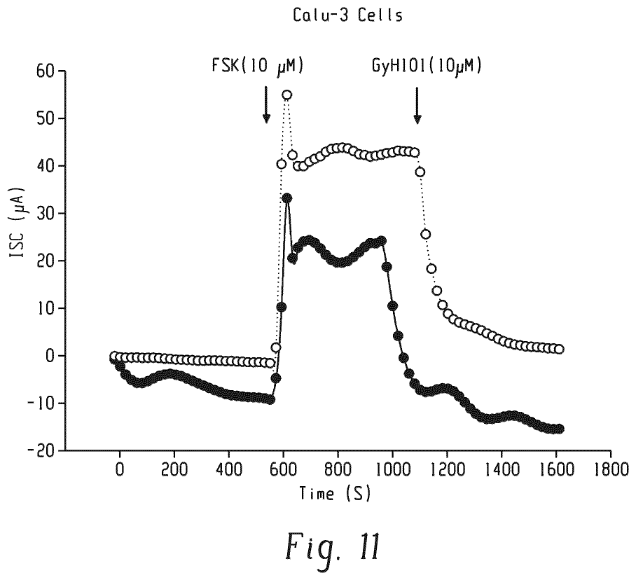

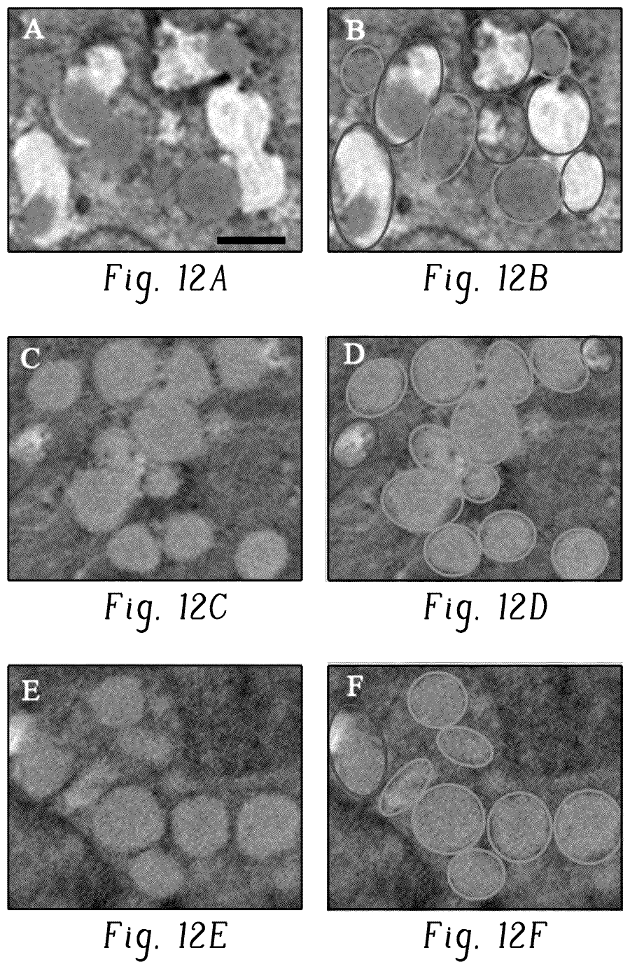

For its part, FIG. 12 demonstrates CFTR inhibitors 172 and GlyH-101 inhibit forskolin-stimulated secretion of intra-vesicular mucin from Calu-3 cells. A and B are from control cells, C and D are from cells exposed to 172, and E and F are from cells exposed to GlyH-101. The vesicles outlined in red are the partial/empty vesicles and those outlined in green are the filled vesicles. Scale bar=500 nm.

TABLE 6
MAJOR PROTEINS IDENTIFIED IN CALU-3 CELL POROSOME PROTEOME
BY LC-MS/MS AND WESTERN BLOT [*] ANALYSIS

| | | | | Calu-3 |
|---|---|---|---|---|
| | | | | porosome |
| | | | | proteins |
| Gene | | | Calu-3 cell porosome proteins identified using | identified using |
| symbol | MW | | LC-MS/MS | western blots |
| ACTB | 42 | kDa | Actin, cytoplasmic 1 | * |
| VIME | 54 | kDa | Vimentin | |
| ANXA2 | 39 | kDa | Annexin 2A | |
| FLNA | 283 | kDa | Filamin-A | |
| GNAI | 40 | kDa | Guanine nucleotide binding protein G(i) alpha | * |
| | | | inhibiting activity | |
| TBB5 | 50 | kDa | Tubulin beta chain | |
| TBA1A | 50 | kDa | Tubulin alpha-1A chain | |
| STX1A | 33 | kDa | Syntaxin-1A | * |
| PROF1 | 15 | kDa | Profilin-1 | |
| VDAC1 | 31 | kDa | Voltage-dependent anion-selective channel | |
| | | | protein 1 | |
| EZRI | 69 | kDa | Ezrin | |
| SPTN1 | 285 | kDa | Spectrin alpha chain | |
| SPTB2 | 275 | kDa | Spectrin beta chain | |
| ANXA3 | 36 | kDa | Annexin A3 | |
| TAGL2 | 23 | kDa | Transgelin-2 | |
| VDAC3 | 31 | kDa | Voltage-dependent anion-selective channel | |
| | | | protein 3 | |
| CLIC1 | 27 | kDa | Chloride intracellular channel protein 1 | |
| RAB1A | 23 | kDa | Ras-related protein Rab-1A | |
| RAB3C | 26 | kDa | Ras-related protein Rab-3C | |
| MYL6 | 17 | kDa | Myosin light polypeptide 6 | |
| MYH9 | 227 | kDa | Myosin-9 | |
| CALM | 17 | kDa | Calmodulin | |
| RTN4 | 130 | kDa | Reticulon-4 | |
| G3BP1 | 52 | kDa | Ras GTPase-activating protein-binding protein 1 | |
| ARF3 | 21 | kDa | ADP-ribosylation factor 3 | |
| MOES | 68 | kDa | Moesin | |
| COF1 | 19 | kDa | Cofilin-1 | |
| ASAP2 | | | Arf-GAP with SH domain, ANK repeat and PH | |
| | | | domain-containing protein 2 | |
| P4R3A | | | Serine/threonine-protein phosphatase 4 | |
| RASA1 | 117 | kDa | Ras GTPase-activating protein 1 | |
| PTN14 | 136 | kDa | Tyrosine-protein phosphatase non-receptor type | |
| | | | 14 | |
| CFTR | 168 | kDa | Cystic fibrosis transmembrane conductance | |
| | | | regulator | |
| CLC3 | 85 | kDa | Chloride channel protein 3 | * |
| SNP25 | 25 | kDa | Synaptosomal-associated protein 25 | * |

* SNAP-25 immunoisolated porosome complexes were obtained using 1% Triton-Lubrol-solubilized Calu-3 cells.

The presence of the cystic fibrosis transmembrane conductance regulator (CFTR) as a component of the porosome complex in the human airway epithelia is also known. Therefore, the regulation by this porosome-associated CFTR on the quality of mucus secretion via the porosome complex at the cell plasma membrane was hypothesized, and subsequently found to be the case. Those findings have progressed the understanding of CFTR—associated porosome, influencing mucous secretion in lung epithelia, and provide critical insights into the etiology of CF disease.

FIG. 13 presents a transmission electron micrograph of a porosome complex associated with a docked secretory vesicle at the apical end of an exocrine pancreatic acinar cell. (a) Part of the apical end of a pancreatic acinar cell demonstrating within the green bordered square, the presence of a porosome and a docked secretory vesicle called zymogen granule (ZG). Fusion of the docked ZG at the base of the porosome complex forms a fusion pore (FP). The electron dense secretory vesicle of the exocrine pancreas. (Bar=400 nm; FIG. 4a only). (b) The area within the green square in a has been enlarged to show the apical microvilli (MV) and a section through porosome and the ZG. Note the ZG membrane (ZGM) bilayer is attached directly to the base of the porosome cup to form the continuity or FP. (c) A higher magnification of the porosome depicts in further detail the porosome bilayer and cross section through the three protein rings, with the thicker ring (blue arrowhead) present close to the opening of the porosome complex to the outside. The third and the lowest ring away from the porosome opening is docked and fused with the ZGM. (d) Yellow outline of the porosome membrane is shows for further clarity. The porosome membrane is in continuity with the apical plasma membrane (PM) at the apical end of the pancreatic acinar cell facing the lumen (L), and also defines the exact points of contact and fusion of the ZGM at the base of the porosome membrane, to form the FP (12).

Turning to FIG. 14 there is presented a transmission electron micrograph of insulin-secreting Min6 cells (endocrine pancreatic beta-cell) demonstrating the presence of porosomes at the cell plasma membrane (a) and a porosome associated with a docked secretory vesicle (b) at the apical end of a cell. Clathrin coated vesicle is shown (c) which differs from the cup-shaped porosome complex. Isolated porosomes measure on average 91 nm, as also demonstrated using photon correlation spectroscopy (d).

Therefore, in view of the presence of porosomes in endothelial cell structures, the techniques taught herein for porosome structure alteration, small molecule targeting, or reconstitution apply to airway cells. Likewise, as airway passages are exposed to the air, drug delivery methods such as nebulizers, inhalers, atomizers and the like such as are known in the art are contemplated for delivery of any of the treatments taught herein.

### Porosome Reconstitution

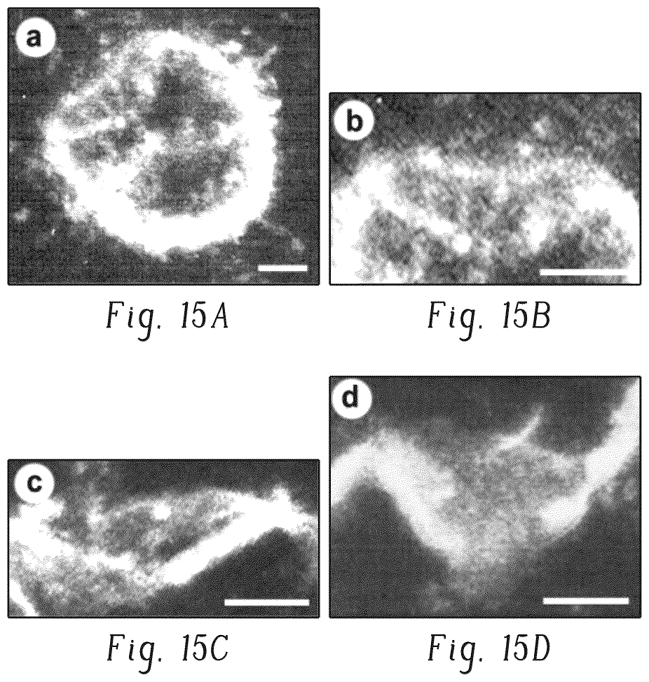

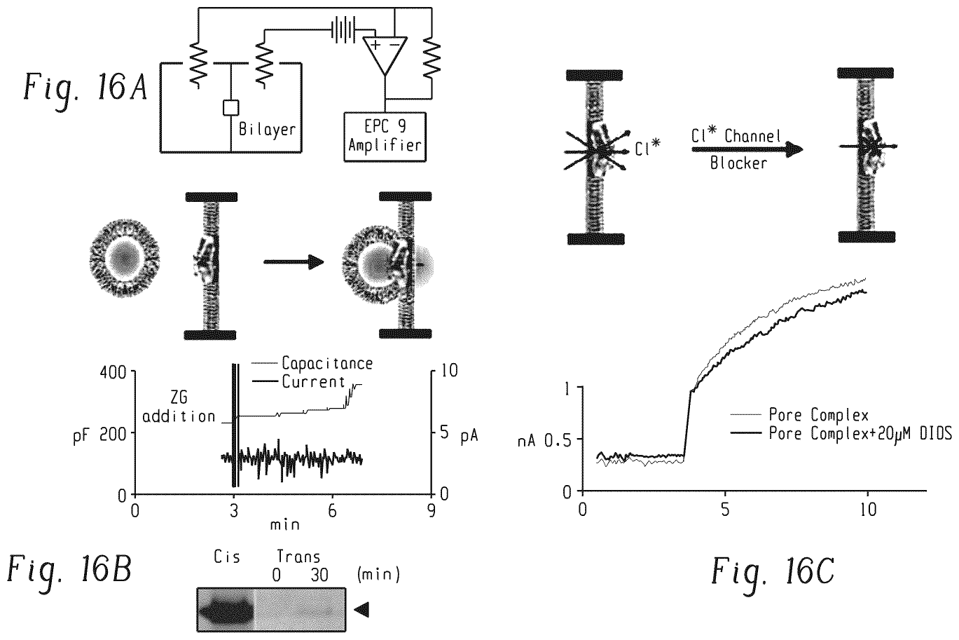

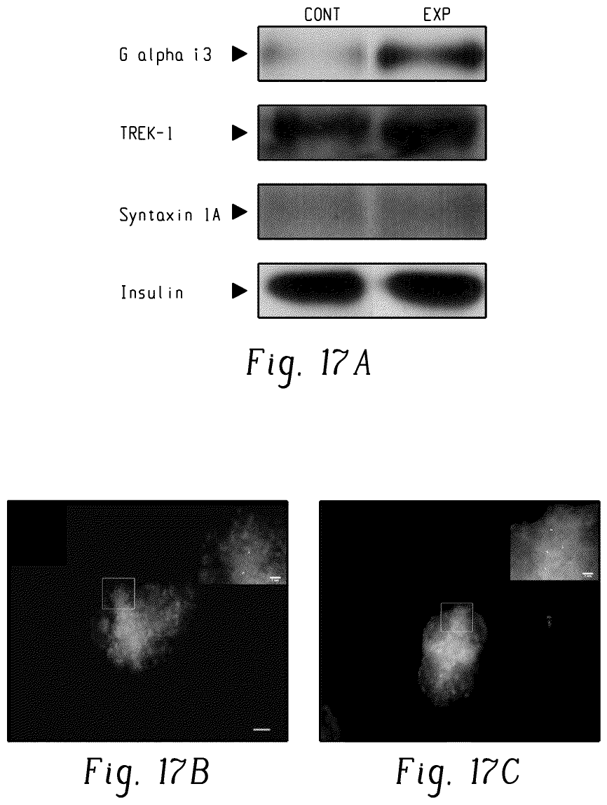

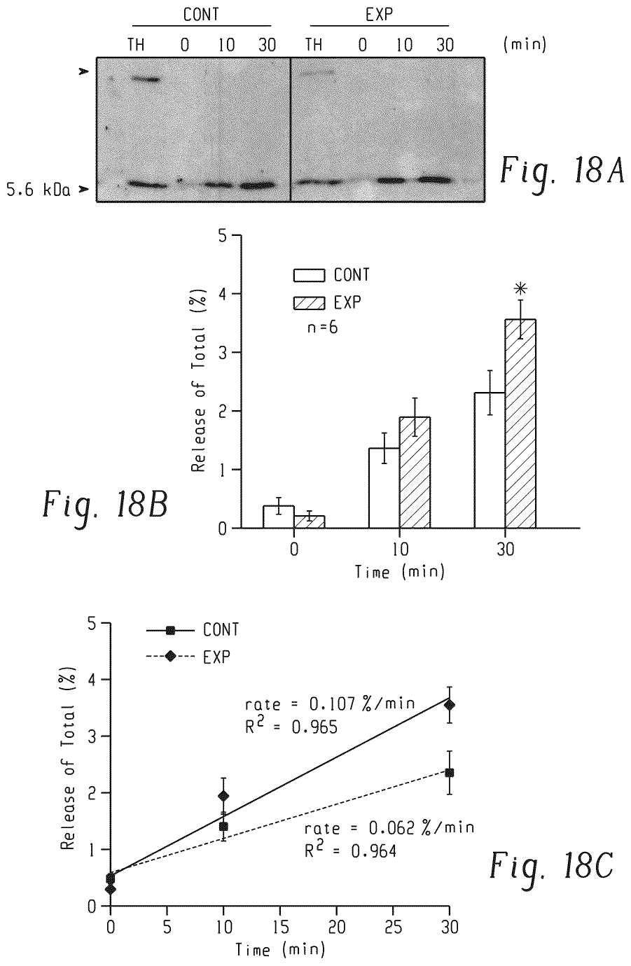

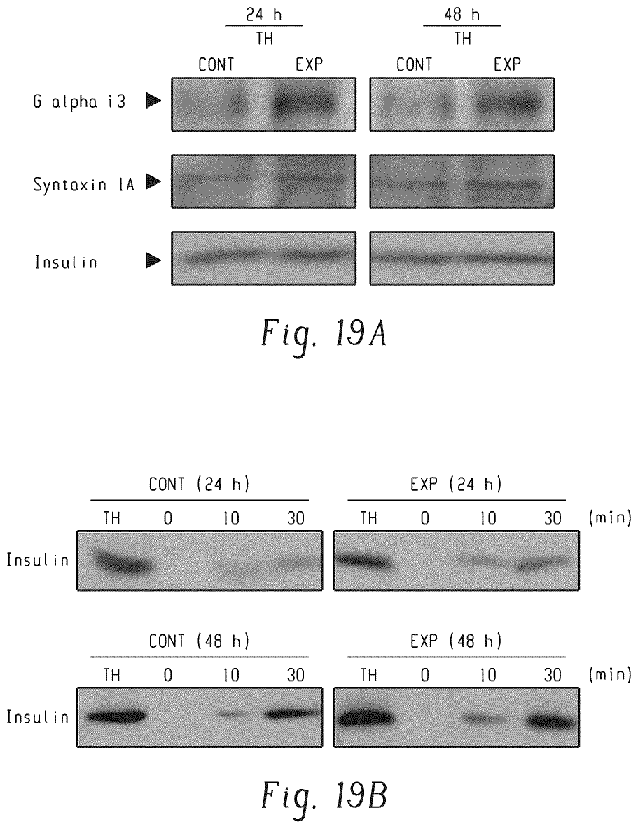

Illustrated below in further detail, porosomes have been functionally reconstituted into both artificial lipid membranes (FIGS. 15 and 16) and into live cells (FIGS. 17 and 18). Furthermore, porosomes reconstituted into live cells are stable and functional (FIG. 19). This powerful porosome reconstitution capability provides a therapeutic approach in the treatment of the CF disease.

FIG. 15 presents electron micrographs of reconstituted exocrine pancreatic porosome complexes in liposomes. The porosomes demonstrate a cup-shaped basket-like morphology. (a) A 500-nm lipid vesicle with an incorporated porosome isolated from the exocrine pancreas is shown. The reconstituted complex at greater magnification is shown in b-d. Bar=100 nm.

Turning to FIG. 16 there is illustrated that a lipid bilayer-reconstituted porosome complex from the exocrine pancreas is functional. (a) Presents a schematic drawing of the bilayer setup for electrophysiological measurements. (b) Illustrates zymogen granules (ZGs) added to the cis side of the bilayer fuse with the reconstituted porosomes, as demonstrated by an increase in capacitance and current activities, and a concomitant time dependent release of amylase (a major ZG content) to the trans side of the membrane. The movement of amylase from the cis to the trans side of the chamber was determined by immunoblot analysis of the contents in the cis and the trans chamber over time. (c) As demonstrated by immunoblot analysis of the immunoisolated complex, electrical measurements in the presence and absence of chloride ion channel blocker DIDS, demonstrate the presence of chloride channels in association with the complex.

FIG. 17 illustrates the enriched presence of TREK-1, Gi3, and Syntaxin-1A immunoreactivity in porosome reconstituted insulin-secreting Min6 cells. A Western blot analysis of 5 μg of Min6 cell homogenate from control and porosome-reconstituted cells. Note the enriched presence of all 3 porosome proteins: TREK-1, Gi3, and Syntaxin-1A. No change in insulin immunoreactivity is observed in the reconstituted Min6 cell homogenate. B, Immunofluorescence microscopy demonstrates increased SNAP-25 (green) and Gi3 (red) immunoreactivity and their increased colocalized presence in porosome reconstituted Min6 cells. Data represents 1 of 4 similar experiments. Scale bar (inset) a and b, 20 μm (13).

FIG. 18 presents insulin-secreting porosomes reconstituted in live Min6 cells, demonstrate elevated glucose-stimulated insulin secretion. Note the increase in time-dependent insulin release from reconstituted Min6 cells. A Representative insulin immunoblot of total Min6 cell homogenate (TH) and glucose-stimulated insulin release at times 0, 10, and 30 minutes, in control and porosome reconstituted experimental Min6 cells. A preproinsulin band is present only in the TH fraction and not in the secreted fraction. B, Bar graph of percent insulin release at time 0, 10, and 30 minutes, in control and reconstituted experimental Min6 cells. A significant increase in time-dependent insulin release from porosome-reconstituted Min6 cells is observed in the 30-minute time point (n=6; *P<0.05). Note, no change in basal insulin release is observed in porosome-reconstituted Min6 cells. C, The rate of insulin secretion per minute was calculated to be 0.062%/min of the total in control cells that increased to 0.107%/min of the total in the porosome-reconstituted cells, a 70% increase in the insulin release rate.

FIG. 19 illustrates the enriched presence of Gαi3, and Syntaxin-1A immunoreactivity in homogenates of porosome-reconstituted Min6 cells, and consequent glucose-stimulated insulin release observed at 24 and 48 hours after reconstitution. A, Representative Western blots of Min6 cell homogenate from control and porosome-reconstituted (experimental) Min6 cells at 24 and 48 hours respectively, demonstrating the enriched presence of the porosome proteins Gi3, and Syntaxin-1A. No change in total insulin immunoreactivity is detected in the experimental homogenate. B, The enriched presence of porosome proteins in A is reflected on the elevated levels of glucose-stimulated insulin release in both the 24 and 48 hours after porosome reconstitution into live Min6 cells. Results represent 1 of 3 separate experiments.

Mucin stored in secretory vesicles in mucin-secreting cells of the airways epithelia occurs either by complete collapse of the vesicle membrane at the cell plasma membrane, or the transient fusion of secretory vesicles at the base of plasma membrane-associated porosomes. The functional reconstitution of the insulin-secreting porosome complex in live β-cells of the endocrine pancreas, opens a window into the treatment of cystic fibrosis and type 1 diabetes.

Examples of insulin secreting porosome proteins in the human airway epithelia include those as listed in below Table 7. Embodiments of the invention can include one or more identified small molecules that directly act upon one or more of the above proteins to affect mucin secreting porosome structure and/or function.

TABLE 7
MAJOR PROTEINS IDENTIFIED IN CALU-3 CELL POROSOME PROTEOME
BY LC-MS/MS AND WESTERN BLOT [*] ANALYSIS.

| | | | | Calu-3 porosome |
|---|---|---|---|---|
| Gene | | | Calu-3 cell porosome proteins identified | proteins identified |
| symbol | MW | | using LC-MS/MS | using western blots |
| ACTB | 42 | kDa | Actin, cytoplasmic 1 | * |
| VIME | 54 | kDa | Vimentin | |
| ANXA2 | 39 | kDa | Annexin 2A | |
| FLNA | 283 | kDa | Filamin-A | |
| GNAI | 40 | kDa | Guanine nucleotide binding protein G(i) | * |
| | | | alpha inhibiting activity | |
| TBB5 | 50 | kDa | Tubulin beta chain | |
| TBA1A | 50 | kDa | Tubulin alpha-1A chain | |
| STX1A | 33 | kDa | Syntaxin-1A | * |
| PROF1 | 15 | kDa | Profilin-1 | |
| VDAC1 | 31 | kDa | Voltage-dependent anion-selective channel | |
| | | | protein 1 | |
| EZRI | 69 | kDa | Ezrin | |
| SPTN1 | 285 | kDa | Spectrin alpha chain | |
| SPTB2 | 275 | kDa | Spectrin beta chain | |
| ANXA3 | 36 | kDa | Annexin A3 | |
| TAGL2 | 23 | kDa | Transgelin-2 | |
| VDAC3 | 31 | kDa | Voltage-dependent anion-selective channel | |
| | | | protein 3 | |
| CLIC1 | 27 | kDa | Chloride intracellular channel protein 1 | |
| RAB1A | 23 | kDa | Ras-related protein Rab-1A | |
| RAB3C | 26 | kDa | Ras-related protein Rab-3C | |
| MYL6 | 17 | kDa | Myosin light polypeptide 6 | |
| MYH9 | 227 | kDa | Myosin-9 | |
| CALM | 17 | kDa | Calmodulin | |
| RTN4 | 130 | kDa | Reticulon-4 | |
| G3BP1 | 52 | kDa | Ras GTPase-activating protein-binding | |
| | | | protein 1 | |
| ARF3 | 21 | kDa | ADP-ribosylation factor 3 | |
| MOES | 68 | kDa | Moesin | |
| COF1 | 19 | kDa | Cofilin-1 | |
| ASAP2 | | | Arf-GAP with SH domain, ANK repeat and | |
| | | | PH domain-containing protein 2 | |
| P4R3A | | | Serine/threonine-protein phosphatase 4 | |
| RASA1 | 117 | kDa | Ras GTPase-activating protein 1 | |
| PTN14 | 136 | kDa | Tyrosine-protein phosphatase non-receptor | |
| | | | type 14 | |
| CFTR | 168 | kDa | Cystic fibrosis transmembrane conductance | |
| | | | regulator | |
| CLC3 | 85 | kDa | Chloride channel protein 3 | * |
| SNP25 | 25 | kDa | Synaptosomal-associated protein 25 | * |

* SNAP-25 immunoisolated porosome complexes were obtained using 1% Triton-Lubrol-solubilized Calu-3 cells.

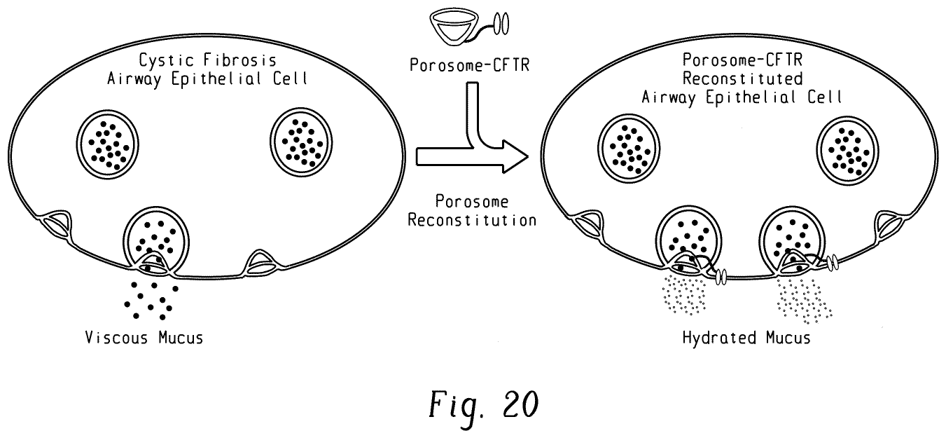

Embodiments of the invention provide an approach for large-scale isolation of mucin-secreting porosome complex from non-CF affected normal human airway epithelia and reconstitution of the porosome complex into CF affected airway epithelia to restore porosome function. FIG. 20 presents an example schematic drawing of functional mucin-secreting porosome complex into the airway epithelia cells in CF patients and amelioration of the CF disease. The presence of the CFTR complex as part of the overall porosome structure is denoted with a linker line which, while not positionally accurate, is merely present to highlight the presence of this particular sub-component of the overall porosome structure. In some embodiments the CRISPR knockout of porosome proteins one at a time, is used to determine what other proteins within the complex are lost at the porosome complex in addition to the knockout protein. Those proteins that are lost from the porosome complex are the ones associated with each other and the knockout protein within the complex. Such systematic studies enable the deciphering of the distribution of all proteins within the porosome complex and help in targeting specific proteins within the complex to modulate and ameliorate secretory function and correct secretory defects and the resulting disease. Furthermore, since the airway epithelia cells are a terminally differentiated population, their average half-life is 6 months in the trachea and more than 18 months in the lung, making them ideal targets for CF porosome-reconstitution therapy.

In some embodiments, Calu-3 and other suitable human airway epithelial cell lines are chosen for porosome harvest for therapy: Human airway epithelial cell lines are used to isolate human mucin-secreting porosome complex in CF therapy. For example, Calu-3 cells were grown in Dulbecco's Modified Eagle Medium: Nutrient Mixture F-12 (DMEM/F-92 12) containing 15% fetal bovine serum. Cells were incubated in a humidified 93% atmosphere at 37° C. and 5% CO2. The Calu-3 cells were the source of the mucin-secreting porosome isolated for CF study and therapy. Mucin-secreting porosome complexes were isolated from Calu-3 cells using SNAP-25-specific antibody conjugated to protein A-Sepharose® (an affinity resin for immunoprecipitation and antibody purification procedures). Calu-3 cells were solubilized using a solubilization buffer composed of 2% Triton X-100 (2-[4-(2,4,4-trimethylpentan-2-yl) phenoxy]ethanol, a nonionic surfactant), 1 mM benzamidine, 5 mM Mg-ATP, and 5 mM EDTA in PBS (pH 7.4), supplemented with protease inhibitor mix. Each immune isolation used a scale-up and modification as required of the following protocol: 2 mg of Triton-solubilized Calu-3 cells, 5 ug of SNAP-25 antibody conjugated to protein A-Sepharose® beads and incubated for 1 hour on ice, followed by 3 washes of 10 volumes of wash buffer (500mMNaCl,10mM Tris, and 2 mM EDTA; pH7.5). The immune-pull down complex associated with the immune-Sepharose® beads was eluted using low pH (pH 3) PBS to dissociate the porosome complex from the antibody bound to the beads, and the eluted sample was immediately returned to neutral pH in a total volume of 200 μL. Various dilutions of the isolated complex were tested for optimal reconstitution into the CF epithelia in human lung organoid, prior to animal, and/or human clinical trials. Isolated porosome suspensions were administered and tested in animal models of the CF disease using a nebulizer for uniform distribution into the airway epithelia and lung. Such systematic studies allowed the ability to optimize the functional reconstitution of isolated porosome complexes and help in ameliorating secretory function and correct secretory defects and the resulting CF disease. We further note that no additional steps were necessary to ensure the correct insertion or orientation of the porosome complex into the epithelial cells when administered via nebulization.

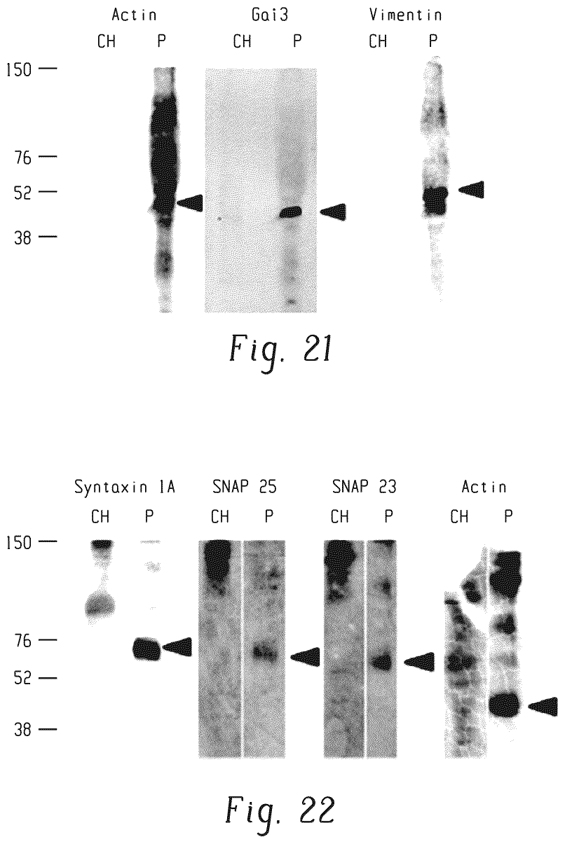

In some embodiments, since Gαi3 is present in the porosome complex of Calu-3 cells (illustrated in FIG. 21) and previous studies report that “pertussis toxin which uncouples GTP-binding G proteins from their receptors, and guanosine 5′[beta-thiophosphate], which prevents G proteins from interacting with their effector proteins, increase Cl− currents in airway epithelia isolated from CF patients” (PNAS 1992, 89(22): 10623-10627), suggests the targeting of the Gαi3 in the porosome complex, resulting in the restoration of cAMP-activated Cl-currents and normal mucin secretion in the airway epithelia of CF patients.

In some embodiments, vimentin is present in the porosome complex in mucin-secreting cells of the human epithelia. As seen in FIG. 21 which illustrates an immunoblot analysis of the total Calu-3 cell homogenate (CH) and isolated porosome complex (P), demonstrates the presence of porosome proteins actin, Gαi3, and vimentin. Note the enriched presence of the proteins in the porosome complex. Since earlier studies report that “4 beta-phorbol 12-myristate 13-acetate (PMA)-mediated phosphorylation of vimentin, appears to be an intermediate step in PKC stimulation of glycoconjugate secretion is impaired in CF disease.” (Am J Physiol. 1994, 266(3pt1) C611-C621), suggests the targeting of vimentin to modulate Vimentin's activity in the mucin-secreting porosome complex in the airway epithelia, resulting in the restoration of normal mucin secretion in the airway of CF patients. Furthermore, it is also known that CFTR has a regulatory domain that is a substrate to both protein kinases A (PKA) and C (PKC).

FIG. 22 illustrates an immunoisolated CFTR complex using the CFTR-specific antibody, results in pull-down of poro some associated proteins such as syntaxin-1A (present as 70 kDa t-/v-SNARE complex), SNAP-25 (present as 70 kDa t-/v-SNARE complex), SNAP-23 (present as 68 kDa t-/v-SNARE complex), and actin. Thus, in some embodiments, since actin is present in the porosome complex in mucin-secreting cells of the human epithelia, and since earlier studies report that “4 beta-phorbol 12-myristate 13-acetate (PMA)-mediated phosphorylation of vimentin, appears to be an intermediate step in PKC stimulation of glycoconjugate secretion is impaired in CF disease.” (Am J Physiol. 1994, 266(3pt1) C611-C621), thus, this suggests the targeting of vimentin to modulate its activity in the mucin-secreting porosome complex in the airway epithelia, will result in the restoration of normal mucin secretion in the airway of CF patients.

In some embodiments, since CFTR also regulates several other transport proteins, including K+ channels, aquaporin water channels, anion exchangers, the membrane fusion protein syntaxin-1A, and sodium bicarbonate transporters, suggests the targeting and modulating their activity using small molecules in the mucin-secreting porosome complex in the airway epithelia, will result in the restoration of normal mucin secretion in the airway of CF patients.

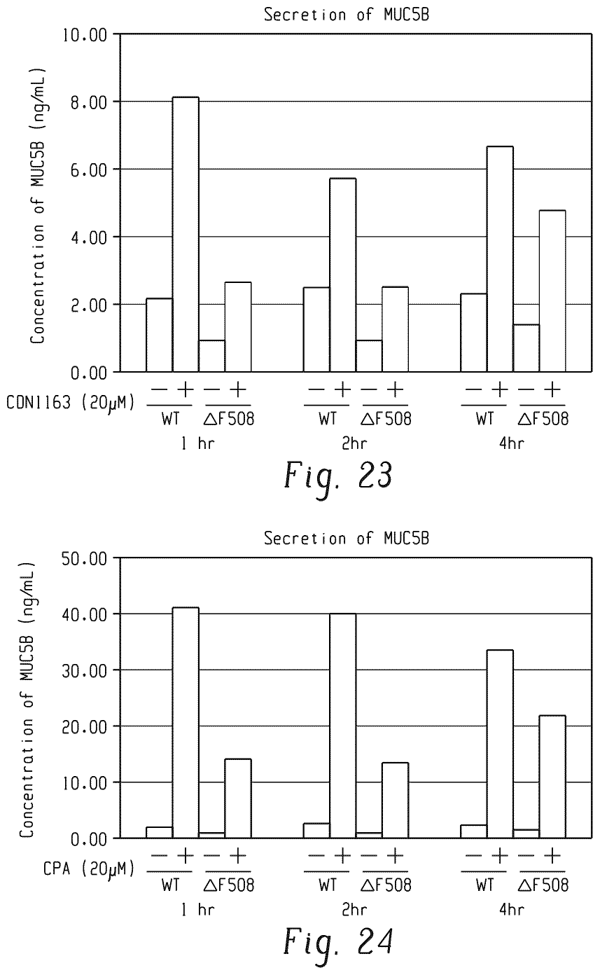

FIG. 23 illustrates the restoration of mucus secretion by CDN1163 in ΔF508-CFTR Human CF Bronchial Epithelial Cells. In mucus secretion in the human air ways epithelium, CFBE41o-6.2 WT-CFTR Human CF Bronchial Epithelial Cell Line and CFBE41o-Human CF Bronchial Epithelial Cell Line (ΔF508-CFTR) were exposed to CDN1163 [4-(1-methylethoxy)-N-(2-methyl-8-quinolinyl)-benzamide], an allosteric Ca2+-ATPase (SERCA) activator. FIG. 23 demonstrates that CDN1163 restores mucus secretion in the CFBE41o-Human CF Bronchial Epithelial Cells (ΔF508-CFTR) to normal levels within an hour. Note that the basal level of Muc5B secretion in the CFBE41o-Human CF Bronchial Epithelial Cells (ΔF508-CFTR) is nearly half that in the normal wild type (WT) cells. CDN1163, the Ca2+ ATPase (SERCA) activator, restores within an hour, the secretion of mucus (Muc5B) in CF bronchial epithelial ΔF508 cells. CDN1163 [4-(1-methylethoxy)-N-(2-methyl-8-quinolinyl)-benzamide], therefore, can be used as a small molecule therapy to restore normal mucus function in CF patients. Dosage concentrations can range from 1 nM (nanomolar) to 20 μM (micromolar) as measured via in-subject tissue concentrations. Such techniques for measurement may include blood draws with subsequent analysis, breathalyzer analysis, urinalysis, biopsy, and other techniques such as are known to those skilled in the art.

Similar to FIG. 23, FIG. 24 illustrates increased mucus secretion by alpha-CPA in ΔF508-CFTR Human CF Bronchial Epithelial Cells. CFBE41o-6.2 WT-CFTR Human CF Bronchial Epithelial Cell Line and CFBE41o-Human CF Bronchial Epithelial Cell Line (ΔF508-CFTR) were exposed to Cyclopiezomic Acid (alpha-cycloplazonic acid or α-CPA), an inhibitor of Ca2+-ATPase (SERCA). Results from our study demonstrate that α-CPA stimulates mucus secretion in the CFBE41o-Human CF Bronchial Epithelial Cells (ΔF508-CFTR) within an hour. Note that the basal level of Muc5B secretion in the CFBE41o-Human CF Bronchial Epithelial Cells (ΔF508-CFTR) is nearly half that in the normal wild type (WT) cells. α-CPA, the Ca2+ATPase (SERCA) inhibitor, dramatically (over 10-fold) stimulates within an hour, the secretion of mucus (Muc5B) in CF bronchial epithelial ΔF508 cells. alpha-CPA, and small molecules with similar binding epitopes such as leflunomide, teriflunomide, tolvaptan, conivaptan, omeprazole, lansoprazole, rufinamide, prazosin, terazosin and roflumilast, currently being used for different indications, can therefore be used as a small molecule therapy to restore normal mucus function in CF patients. Data depicted below.

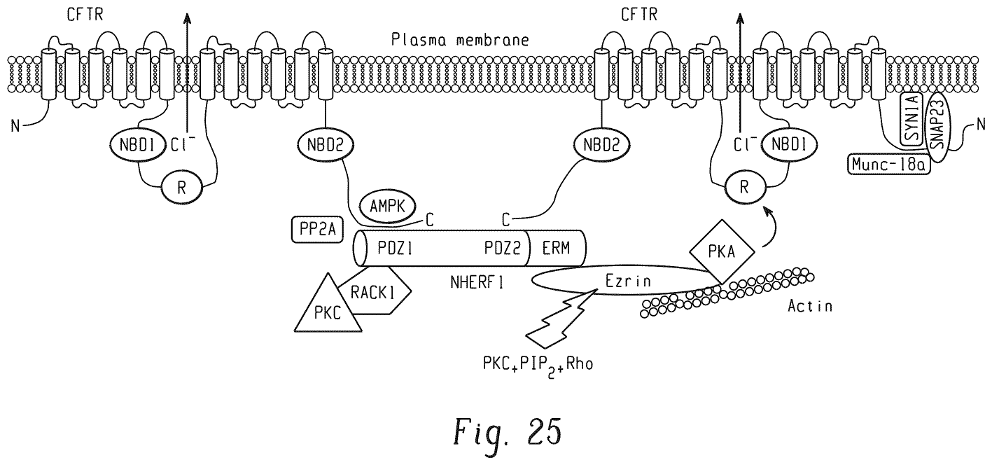

FIG. 25 illustrates a schematic drawing demonstrating some interactions between CFTR and associated proteins (Nature Reviews Mol. Cell Biol. 2006, 7:426-436). Besides mediating the secretion of Cl−, CFTR also regulates several other transport proteins including K+ channels, aquaporin water channels, anion exchangers, the membrane fusion protein syntaxin-1A, and sodium bicarbonate transporters. Hence, in some embodiments, since K+ channels are present in the mucin-secreting porosome complex, and hydration of mucus is a problem in the CF disease, the interactions between the K+ channels, aquaporin water channels and CFTR can be targeted using small molecules such as those described herein.

In cystic fibrosis, since bacterial pathogens contribute to mucus hypersecretion through mobilization of intracellular Ca2+, and Ca2+-ATPase (SERCA) has been identified in the insulin-secreting porosome complex , suggested a role of this ion channel in the secretion of mucus in human airway epithelia.

CDN1163 is an allosteric sarco/endoplasmic reticulum Ca2+-ATPase (SERCA activator that improves Ca2+ homeostasis. Its formal name is 4-(1-methylethoxy)-N-(2-methyl-8-quinolinyl)-benzamide with a CAS Number: 892711-75-0; a molecular formula of: C20H20N2O2; and a formula weight of: 320.4.

CDN1163 attenuates diabetes and metabolic disorders, IC50 & Target: SERCA. In Vitro: CDN1163 (5.5-25 mM; 0-8 hours; rat cardiac myocyte cells) treatment reduces high glucose-induced resistin and nuclear NFATc expression and increases the phosphorylation of AMPKα in a time-dependent manner. In Vivo: CDN1163 (50 mg/kg; intraperitoneal injection; for 5 days to male ob/ob mice and lean ob/+ mice) increases SERCA2 Ca2+-ATPase activity, decreases endoplasmic reticulum (ER) stress-induced cell death in vitro and improves liver Ca2+ transport activity. CDN1163 reduces blood glucose levels and improves metabolic parameters and gluconeogenic gene expression, reverses hepatic steatosis, inhibits ER stress and ER stress-induced apoptosis, and improves mitochondrial efficiency in ob/ob mice in vivo.

Embodiments of the above invention may be used singly, or in combination with each other. By way of non-limiting example, small molecules targeting the porosome complex or portions thereof may be used in combination with the reconstitution of porosomes as above described. Likewise, patients undergoing treatment for a neurological disease as above-described may also be undergoing a similar treatment for an insulin-secretion defect such as diabetes. While certain embodiments of the present invention are above disclosed in connection with treatments/therapies for cystic fibrosis, the present invention is not intended to be so limited in this regard. In particular, it is contemplated that the therapies and treatments disclosed herein may be utilized in the treatment of any secretory diseases, cystic fibrosis and type-1 diabetes being only two of them. In an embodiment, the methods of the present invention may, for example, also be utilized in the treatment of chronic obstructive pulmonary disease (COPD)

### Synthesis Of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics. Identification of chemicals may be through multiple names and nomenclatures including standard IUPAC nomenclature; CAS number; written formula; bond diagram, etc.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In some embodiments, the compounds or small molecules identified are purchased from a variety of vendors, including Sigma-Aldrich, Acros, Fisher, Fluka, Santa Cruz, CombiBlocks, BioBlocks, and Matrix Scientific.

### Cells, Analytical Techniques, and Instrumentation

In certain embodiments, also described herein are methods for profiling porosomes to determine a reactive or modulating molecule. In some instances, the methods comprise profiling a porosome containing cell sample or a porosome containing cell lysate sample. In some embodiments, the cell sample or cell lysate sample is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g., Danino rerio). In some cases, the worm cell is from a nematode (e.g., C. elegans). In some cases, the amphibian cell is from a frog. In some embodiments, the arthropod cell is from a tarantula or hermit crab.

In some embodiments, the porosome complex isolated from cells or cell lysate sample is obtained from a mammalian cell. In some instances, the mammalian cell is an epithelial cell, connective tissue cell, hormone secreting cell, a nerve cell, a skeletal muscle cell, a blood cell, or an immune system cell.

Exemplary mammalian cells include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, the porosome containing cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the porosome containing cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the porosome containing cell sample or cell lysate sample is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Lyl, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the porosome containing cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the porosome containing cell sample or cell lysate sample is obtained from an individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, would be examined under a microscope.

### Sample Preparation and Analysis

In some embodiments, a porosome containing sample solution comprises a cell sample, a cell lysate sample, or a sample comprising isolated proteins. In some instances, the sample solution comprises a solution such as a buffer (e.g., phosphate buffered saline) or a media. In some embodiments, the media is an isotopically labeled media. In some instances, the sample solution is a cell solution.

In some embodiments, the porosome containing solution sample (e.g., cell sample, cell lysate sample, or comprising isolated proteins) is incubated with a compound for analysis of protein-probe interactions. In some instances, the solution sample (e.g., cell sample, cell lysate sample, or comprising isolated proteins) is further incubated in the presence of an additional compound probe. In other instances, the solution sample (e.g., cell sample, cell lysate sample, or comprising isolated proteins) is further incubated with a ligand. In such instances, the solution sample is incubated with a probe and a ligand for competitive protein profiling analysis.

In some cases, the porosome containing cell sample or the cell lysate sample is compared with a control. In some cases, a difference is observed between a set of probe protein interactions between the sample and the control. In some instances, the difference correlates to the interaction between the small molecule and one or more porosome proteins.

In some embodiments, one or more methods are utilized for labeling a porosome containing solution sample (e.g., cell sample, cell lysate sample, or comprising isolated proteins) for analysis of probe protein interactions. In some instances, a method comprises labeling the sample (e.g., cell sample, cell lysate sample, or comprising isolated proteins) with an enriched media. In some cases, the sample (e.g., cell sample, cell lysate sample, or comprising isolated proteins) is labeled with isotope-labeled amino acids, such as 13C or 15N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments a method comprises incubating a solution sample, or a porosome sample mixture, (e.g., cell sample, cell lysate sample, or comprising isolated proteins) with a labeling group to tag one or more proteins of interest for further analysis. The labeling group can be an isotopically labeled group such an amino acid or acid enriched in a 13C, 15N, or deuterium, or may comprise a molecular label such as biotin, folic acid, luciferase, or an amino acid tag. The label may further comprise a linker that is optionally isotopically labeled. The linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with 13C, 15N, and/or deuterium atoms at one or more amino acid residue positions within the linker. In some cases, the biotin-linker moiety is an isotopically labeled TEV-tag.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex, or a subsample of thereof, is separated and visualized utilizing an electrophoresis system, such as through a gel electrophoresis, or a capillary electrophoresis. Exemplary gel electrophoresis includes agarose-based gels, polyacrylamide-based gels, or starch-based gels. In some instances, the probe-protein is subjected to a native electrophoresis condition. In some instances, the probe-protein is subjected to a denaturing electrophoresis condition.

In certain embodiments the probe-protein complex is harvested using such standard techniques as are known in the art and dependent upon the specific chemistry of the probe-protein complex. Example techniques can include, at least: tangential flow filtration, chromatography, centrifugation, liquid chromatography, electrophoresis, and the like, either alone or in combination, as are commonly employed to separate a target protein from a mixture.

In some instances, the probe-protein complex after harvesting is further fragmentized to generate protein fragments. In some instances, fragmentation is generated through mechanical stress, pressure, or chemical fragmentation. In some instances, the protein from the probe-protein complexes is fragmented by chemical fragmentation. In some embodiments, the chemical fragmenting agent is a protease.

Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase C1p, Escherichia coli phage K1F endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high-performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more binding proteins or protein fragments interacting with small molecules as described herein. In some embodiments, the NMR method includes one dimensional (1D) NMR methods, two dimensional (2D) NMR methods, solid state NMR methods and NMR chromatography. Exemplary 1D NMR methods include 1Hydrogen, 13Carbon, 15Nitrogen, 17Oxygen, 19Fluorine, 31Phosphorus, 39Potassium, 23Sodium, 33Sulfur, 87Strontium, 27Aluminium, 43Calcium, 35Chlorine, 37Chlorine, 63Copper, 65Copper, 57Iron, 25Magnesium, 199Mercury or 67Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state 13Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification.

In some embodiments, the algorithm comprises ProLuCID algorithm, Probity, Scaffold, SEQUEST, or Mascot.

In some embodiments, a value is assigned to each protein from the probe-protein complex. In some embodiments, the value assigned to each of the protein from the probe-protein complex is obtained from the mass spectroscopy analysis. In some instances, the value is the area-under-the curve from a plot of signal intensity as a function of mass-to-charge ratio. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

By way of example, in certain embodiments of the invention a first isolated porosome sample mixture is created from a human epithelial cell line and a second isolated porosome sample mixture is created from the 508 cystic fibrosis mutant line. Ratios may be made first:second or second:first as long as the ratio is performed consistently across all compared proteins from each sample mixture. In some instances, anything with a ratio of 20% or less at a 95% confidence interval is considered an absent protein. Those of skill in the art may appreciate the need to alter cut-off values for ratios and confidence intervals without departing from the scope of this invention. In certain embodiments of the invention, the second sample mixture may be a “knock out” cell line where one or more porosome proteins are “knocked out” using such techniques as CRISPER.

### Kits/Article of Manufacture

Disclosed herein, in certain embodiments, are kits and articles of manufacture for use to generate a porosome protein adduct or with one or more methods described herein. In some embodiments, described herein is a kit for detecting porosome protein ligand interaction. In some embodiments, such kit includes small molecule ligands, small molecule fragments or libraries, compound probes, and/or controls, and reagents suitable for carrying out one or more of the methods described herein. In some instances, the kit further comprises samples, such as a cell sample, and suitable solutions such as buffers or media. In some embodiments, the kit further comprises recombinant porosome protein or proteins for use in one or more of the methods described herein. In some embodiments, additional components of the kit comprise a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error or within the error expected from manufacturing, production, or experimental tolerances.

Suitable alterations to the above are readily apparent to those of skill in the art and naturally are encompassed and expressly contemplated. For example, normal manufacturing tolerances may induce variances from the above presented formulations without departing from the broader scope of this invention.

Compounds described herein may be formed as, and/or used as, acceptable salts. The type of acceptable salts, include, but are not limited to: (1) acid addition salts, formed by reacting the free base form of the compound with an acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-β-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

The effective dose and method of administration of a particular embodiment of the instant invention may vary based on the individual patient and stage of any present diseases (e.g., influenza, covid, HIV, other co-morbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by an individual physician in view of a patient to be treated. Dosage and administration are adjusted to provide sufficient levels of embodiments of the instant invention to maintain the desired effect (e.g., elimination or reduction of enveloped virus particles or activity in a host). Additional factors that may be taken into account include the severity of any disease state, age, weight, and gender of the patient; diet, time and frequency of the administration, drug combination(s), reaction sensitivities, and tolerance/response to therapy.

Short acting pharmaceutical compositions are administered daily whereas long-acting pharmaceutical compositions are administered every 2, 3 to 4 days, every week, or once every two weeks or more. Depending on half-life and clearance rate of the particular formulation, the pharmaceutical compositions of the instant invention may be administered once, twice, three, four, five, six, seven, eight, nine, ten or more times per day.

Normal dosage amounts for active ingredients may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of the active ingredients described herein are those that provides sufficient quantity to attain a desirable effect, including those above-described effects (e.g., the modulation, activation, or interaction with one or more porosome proteins and/or achievement of an effect on a porosome structure). Accordingly, the dose of the active ingredients preferably produces a tissue or blood concentration of both about 1 to 800 μM. Preferable doses produces a tissue or blood concentration of greater than about 10 μM to about 500 μM. Preferable doses are, for example, the amount of active ingredients required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present invention. A constant infusion of embodiments of the invention can be provided so as to maintain a stable concentration of the therapeutic agents.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The pharmacologically active compounds of this invention can be processed in accordance with conventional methods of pharmacy and good manufacturing practices to produce medicinal agents for administration to patients (e.g., mammals including humans) either prophylactically or as part of a treatment regime.

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A method of identifying protein-protein interaction in a porosome structure, comprising:
a) creating a first porosome sample mixture;
b) incubating the porosome sample mixture with a labeling group generating a probe-protein complex;
c) harvesting the probe-protein complex;
d) fragmenting the probe-protein complex, resulting in protein fragments;
e) analyzing the protein fragments via a proteomic method;
f) identifying one or more proteins in the porosome sample mixture, creating a first identified protein set;
g) assigning a value to each protein in a first identified protein set;
h) performing steps a)-g) on a second sample porosome mixture, obtaining a second value for each protein in a second identified protein set;
i) calculating a ratio between the values between paired proteins in the first and second identified protein sets;
wherein said ratio is determinative of a protein-protein interaction either within or adjacent to a porosome structure.

2. The method of claim 1 wherein the first sample porosome mixture is from a standard, control, or wildtype cell sample and the second sample porosome mixture is from a test cell sample.

3. The method of claim 2 wherein the test cell sample is from a knock-out cell line.

4. A kit configured to contain the materials required to perform the steps of claim 1.

5. The method of claim 1 wherein the proteomic method is at least one selected from the group of: LC, LC-MS, MALDI-TOF, GC-MS, CE-MS, and NMR.

6. The method of claim 1 wherein the value for each protein in the first and second identified protein sets correlates with the reactivity of a Lys residue within a protein.

7. The method of claim 1 further comprising:
j) confirming the specific protein-protein interactions within the porosome complex using a small molecule.

8. The method of claim 7 wherein the confirmation is performed via chemical cross-linkage and subsequent confirmation of linkage via mass spectrometry.

9. The method of claim 1 wherein the first and second sample porosome complex function is examined in an artificial lipid bilayer membrane.

10. A composition, comprising:
an artificial porosome in an artificial lipid bilayer membrane.

11. A method, comprising:
cross-linking an artificial porosome to a nanobody humanized to target and bind to one or more domains of one or more porosome or porosome-associated proteins;
delivering to a subject the cross-linked artificial porosome-nanobody.

12. A composition, comprising:
at least one cross-linking molecule;
at least one small molecule modulator targeted to a porosome protein.

13. The composition of claim 12 wherein the cross-linking molecule is an ELP diblock.

14. The composition of claim 12, further comprising:
a nanobody humanized to target and bind to one or more domains of one or more porosome proteins.

15. A composition comprising:
a humanized nanobody with one or more small molecules targeting the domains of one or more porosome proteins and an artificial cysteine;
said cysteine bound to an ELP diblock;
said ELP diblock bound to pAcF.

16. The composition of claim 15 wherein a drug is attached to the pAcF.

17. The composition of claim 16 wherein the drug attached to the pAcF is doxorubicin.

18. A method, comprising:
extracting porosomes from a non-human source;
reconstituting the extracted porosomes into a human cell.

19. The method of claim 18 wherein the porosomes are extracted from human epithelial cells, and stem cells.

20. The method of claim 18 wherein isolated porosomes are reconstituted into organoids or an artificial lipid bilayer.
